(19) United States
(12) Patent Application Publication
(10) Pub. No.: US 2022/0111201 A1
(43) Pub. Date: Apr. 14, 2022

(54) IDENTIFYING A PRESENCE-ABSENCE STATE OF A MAGNETIC RESONANCE IMAGING SYSTEM

(71) Applicant: INSPIRE MEDICAL SYSTEMS, INC., Golden Valley, MN (US)

(72) Inventors: Kevin Verzal, Lino Lakes, MN (US); David Dieken, Minneapolis, MN (US); John Rondoni, Plymouth, MN (US)

(73) Assignee: INSPIRE MEDICAL SYSTEMS, INC., Golden Valley, MN (US)

(21) Appl. No.: 17/495,646

(22) Filed: Oct. 6, 2021

Related U.S. Application Data
(60) Provisional application No. 63/089,118, filed on Oct. 8, 2020.

(51) Int. Cl. A61N 1/08; G01P 13/00; G01P 15/08; G01R 33/02

(52) U.S. Cl. CPC A61N 1/086; G01R 33/02; G01P 15/08; G01P 13/00

(57) ABSTRACT

A system and/or method involving sensing first data via at least one implantable sensor of an implantable medical device (IMD) system, and identifying a presence-absence state of a magnetic resonance imaging (MRI) system using the first data.

### BACKGROUND

Modern medicine has provided previously unimaginable abilities, such as internal imaging. One type of internal imaging includes magnetic resonance imaging. Other modern technologies include implantable medical devices, some types of which may not be compatible with such internal imaging.

### BRIEF DESCRIPTION OF THE DRAWINGS

FIG. 1 is a flow diagram schematically representing an example method comprising identifying a presence-absence state of a magnetic resonance imaging (MRI) system.

FIGS. 2A-2B are block diagrams schematically illustrating an example implantable medical device (IMD) system.

FIGS. 3A-3C are diagrams schematically representing deployment of an example IMD, which includes an implantable sensor arrangement.

FIGS. 4A-4D are block diagrams, which may comprise part of a flow diagram in an example method.

FIGS. 5A-5F are block diagrams schematically illustrating example IMDs.

FIG. 6 is a block diagram schematically illustrating an example IMD, which includes an acceleration sensor, an MRI-sensitive conductive element, and a Hall effect sensor.

FIG. 7 is a block diagram schematically illustrating an example IMD, which includes an acceleration sensor, an MRI-sensitive conductive element, and a giant magnetoresistance sensor.

FIG. 8 is a block diagram schematically illustrating an example IMD, which includes an acceleration sensor, an MRI-sensitive conductive element, and a reed switch.

FIGS. 9A-9B are block diagrams schematically illustrating example IMDs, which include an acceleration sensor, an MRI-sensitive conductive element, and a biopotential amplifier.

FIG. 10 is a block diagram schematically representing an example sensor type.

FIGS. 11A-11D are block diagrams schematically illustrating an example MRI engine of an IMD system.

FIGS. 12A-12D are diagrams, which may comprise part of a flow diagram in an example method.

FIGS. 13A-13B are flow diagrams, which may comprise part of a flow diagram in an example method.

FIG. 14 illustrates an example pattern of patient-volitional data and the patient non-volitional data.

FIG. 15 is a block diagram, which may comprise part of a flow diagram in an example method.

FIG. 16 is a block diagram schematically representing example data model types.

FIG. 17 is a block diagram schematically representing at least some example known input sources.

FIG. 18 is a diagram schematically representing an example method of constructing a data model for use in later identifying a presence-absence state of an MRI system.

FIG. 19 is a diagram schematically representing an example method of using a constructed data model for identifying a presence-absence state of an MRI system using internal measurements.

FIG. 20 is diagram schematically representing an example method of constructing a data model.

FIG. 21 is a diagram schematically representing an example method of using a constructed data model for identifying a presence-absence state of an MRI system.

FIGS. 22A-22B are block diagrams schematically presenting example IMD systems including an MRI engine.

FIGS. 23-35 are diagrams, which may comprise part of a flow diagram in an example method.

FIG. 36 is a flow diagram schematically representing an example method, which may comprise part of a flow diagram in an example method.

FIG. 37 is a diagram including a front view of an example device (and/or example method) implanted within a patient's body.

FIG. 38 is a diagram schematically representing an example IMD.

FIG. 39A is a block diagram schematically representing an example control portion.

FIG. 39B is a diagram schematically illustrating at least some example arrangements of a control portion.

FIG. 40 is a block diagram schematically representing a user interface.

FIG. 41 is a block diagram which schematically represents some example implementations by which an IMD may communicate wirelessly with external devices outside the patient.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some examples of the present disclosure are directed to devices, systems, and/or methods involving sensing first data via at least one implantable sensor of an implantable medical device (IMD) system and identifying a presence-absence state of a magnetic resonance imaging (MRI) system using the first data.

At least some examples of present disclosure are directed to devices, systems, and methods for controlling at least one function or operation of an IMD system, including an IMD implanted within a patient, in response to the identified presence-absence state of the MRI system. In some examples, one or more sensors implanted in the patient are utilized to sense or detect the first data which is indicative of a presence-absence state of the MRI system. In some examples, the first data includes patient-volitional data (e.g., body motion and posture), and/or patient non-volitional data (e.g., externally induced body motion and/or vibrations), which exhibit a pattern indicative of the presence-absence state of the MRI system. In some examples, the IMD system identifies the presence-absence state of the MRI system prior the MRI system executing an MRI scan on a patient with the IMD implanted in the patient's body. In response to the identified presence-absence state of the MRI system, a feature of the IMD may be controlled, such as disabling or enabling a feature and/or switching the IMD to an MRI mode of operation.

In some examples, the devices, systems, and methods of the present disclosure are configured and used for sleep disordered breathing (SDB) care, such as obstructive sleep apnea (OSA) care, which may comprise monitoring, diagnosis, and/or stimulation therapy. However, in other examples, the system is used for other types of care and/or therapy, including, but not limited to, other types of neurostimulation or cardiac care or therapy. In some examples, such other implementations include therapies, such as but not limited to, central sleep apnea, complex sleep apnea, cardiac disorders, pain management, seizures, deep brain stimulation, and respiratory disorders.

It will be further understood that in some instances, a data model may be used to identify some of the internally sensed inputs and/or some of the ways in which the internally sensed inputs may be used to identify the presence-absence state of the MRI system. Non-data-model techniques may be used with (or without) the data model techniques to determine the desired internally sensed inputs.

Accordingly, it will be further understood that aspects of the various example methods involving non-data models and those involving data models may be selectively mixed and matched with each other as desired to achieve the desired and/or effective manner of identifying the presence-absence state of the MRI system via internally sensed data.

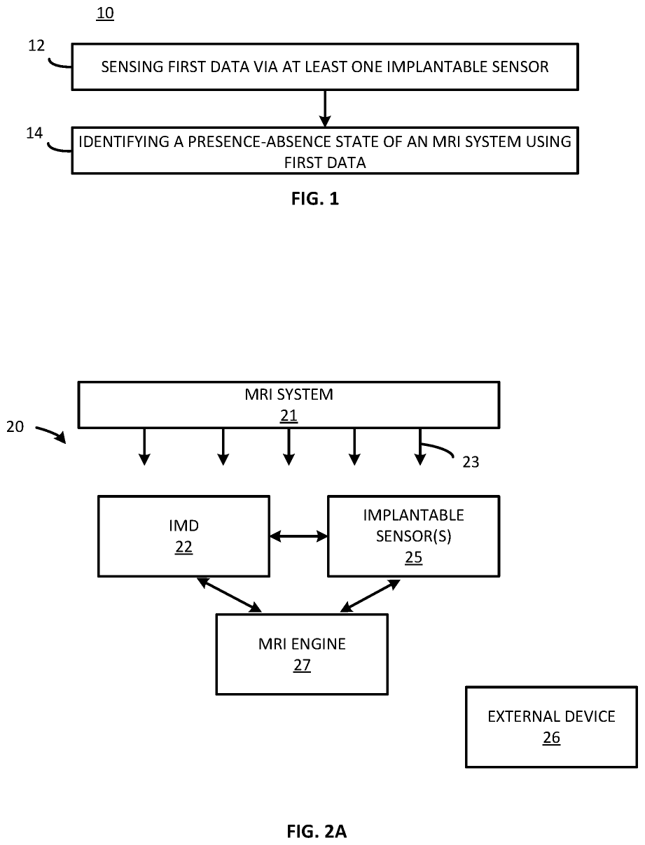

These examples, and additional examples, are described in association with at least FIGS. 1-41.

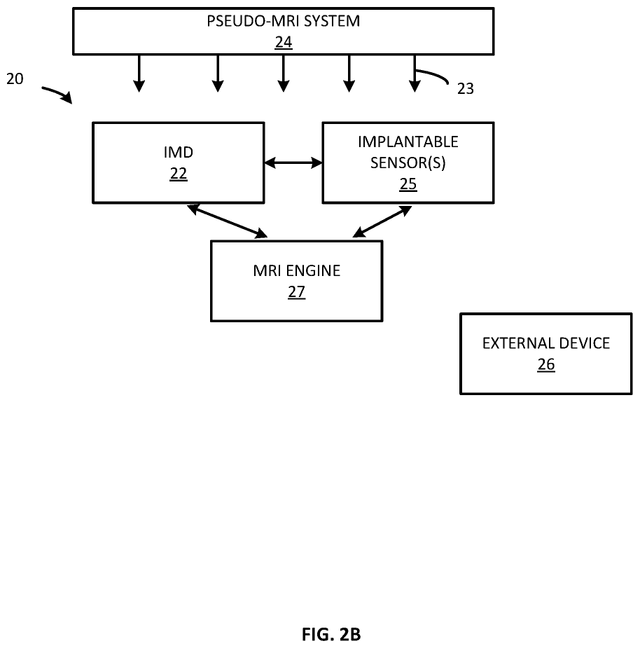

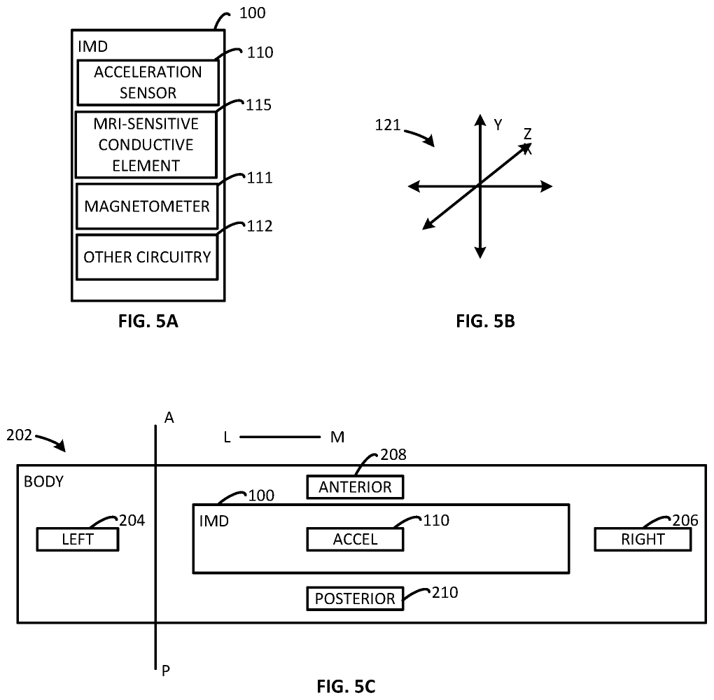

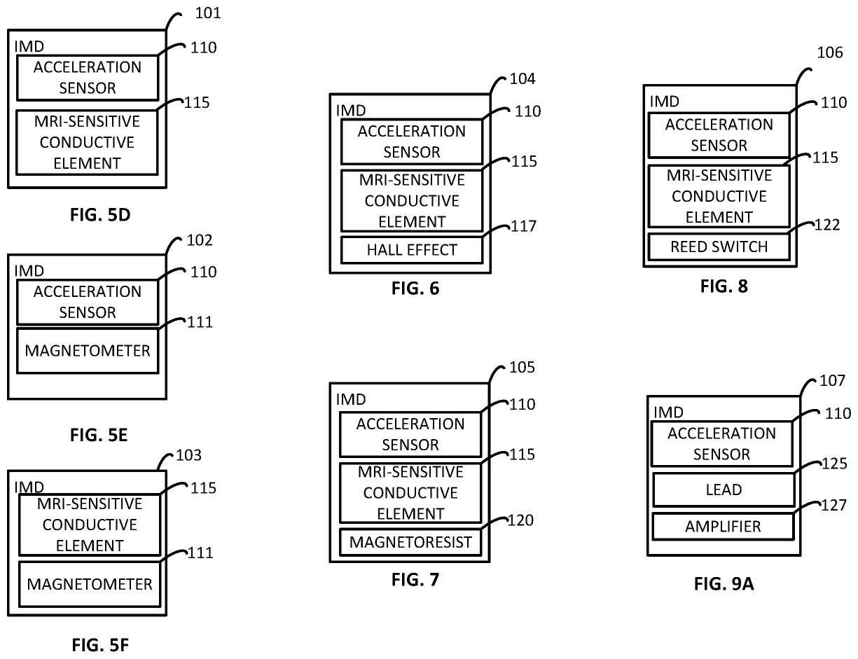

FIG. 1 is a flow diagram schematically representing an example method 10 comprising identifying a presence-absence state of an MRI system. The method 10 includes sensing first data via at least one implantable sensor of an IMD system, as shown at 12 in FIG. 1, and identifying a presence-absence state of an MRI system using the first data, as shown at 14. As further illustrated by FIG. 2A, the IMD system may comprise an IMD and the at least one implantable sensor, which may form part of the IMD or is otherwise in communication with the IMD. The at least one implantable sensor may comprise an acceleration sensor, an MRI-sensitive conductive element, a magnetometer, a giant magnetoresistance sensor, a Hall effect sensor, a reed switch, and/or various combinations therefore, examples of which are further illustrated by at least FIGS. 5A-9B.

As may be appreciated, an MRI system produces MRI fields for scanning a patient to obtain internal images. The MRI fields may comprise at least static magnetic fields and gradient magnetic fields, which may vary over time. For example, MRI systems generally produce three types of electromagnetic fields including static magnetic fields, time-varying gradient magnetic fields, and radio frequency (RF) fields which consist of RF pulses used to produce the internal images. The MRI fields may form a pattern of electromagnetic fields. The static magnetic fields produced by most commercial MRI systems have a magnetic induction ranging from about 0.5 to about 3.0 tesla (T). The frequency of the RF fields used for imaging is related to the magnitude of the static magnetic fields, and, for many MRI systems, the frequency of the RF field ranges from about 6.4 to about 128 megahertz (MHz). The time-varying gradient magnetic field is used in MRI for spatial encoding, and typically has a frequency in the Kilohertz (kHz) range.

A presence-absence state of an MRI system, as used herein, comprises and/or refers to a state indicative of a proximity of the MRI system relative to the IMD. In some examples, the presence-absence state of the MRI system comprises a presence of the MRI system relative to the IMD and/or an absence of the MRI system relative to the IMD. For example, making or declaring a state of a presence of the MRI system may correspond to the IMD being sufficiently close to (e.g., within a threshold distance of) the MRI system such that strong electromagnetic fields are exerted on the IMD by the MRI system. The strong electromagnetic fields may be above a threshold signal strength and may impact the IMD by causing unwanted effects, as further described below. As examples, the threshold signal strength may be above 0.2 T and/or above an electromagnetic strength of electromagnetic fields encountered in day-to-day activity, which may be less than 0.1 T. A state of a presence of the MRI system is generally herein referred to as “a presence of the MRI system” and sometimes interchangeably referred to as “a present state”.

For example, making or declaring a state of an absence of the MRI system may correspond to the IMD being sufficiently far away from (e.g., outside the threshold distance of) the MRI system such that the electromagnetic fields exerted on the IMD by the MRI system are below the threshold signal strength. A state of an absence of the MRI system is generally herein referred to as “an absence of the MRI system” and sometimes interchangeably referred to as “an absent state”.

In some examples, the presence-absence state may be identified as a presence of the MRI system when the individual is physically present with respect to the MRI system and may not be the subject of the MRI scan, but the individual has an implanted IMD which is sensitive to the MRI fields. For example, such individuals may comprise a technician running the MRI scan or a guardian of the subject (e.g., a child) of the MRI scan that is in the room during the MRI scan. As such, a patient, as used herein, is not limited to the subject of the MRI scan, and may comprise any person with an implanted IMD. As further described below, examples are not limited to identifying a presence and/or an absence of the MRI system, and may comprise identifying a non-presence and/or non-absence of the MRI system.

In some examples, the first data sensed by the at least one implantable sensor may include patient-volitional data and/or patient non-volitional data, either of which may be indicative of the presence-absence state of the MRI system. In some examples, the patient-volitional data, as used herein, comprises and/or refers to data caused by or in response to phenomenon that is patient initiated. Example patient-volitional data includes phenomenon, such as body motion and posture, which may occurring during an awake state or which may occur during a sleep state in some instances, as well as other and/or additional physiological data.

In at least some examples, the patient non-volitional data, as used herein, comprises and/or refers to data caused by or in response to phenomenon that is not initiated by the patient, but rather initiated or caused by external elements. Example patient non-volitional data includes phenomenon, such as body motion caused by the MRI system (or other external sources), electromagnetic fields and/or vibrations which are externally induced by the electromagnetic fields. In some examples, at least some of the first data also may comprise data which is not necessarily categorized as either being patient-volitional or patient non-volitional.

As further described herein, the method 10 may include a number of additional steps and/or variations, such as controlling a feature of the IMD in response to the identified presence-absence state of the MRI system. For example, the electromagnetic fields exerted by the MRI system may cause issues for the IMD implanted within the patient, such as power supply issues, false event sensing, and heating and voltage generation on internal components. In various examples, the IMD system may identify the presence-absence state of the MRI system, and optionally, control a particular feature in response to the identified presence-absence state of the MRI system. Controlling the feature (e.g., enabling or disabling) may mitigate or prevent unwanted effects on the IMD from the MRI fields and/or otherwise be used as feedback. A number of IMDs may be designed to switch to an MRI mode in response to a manual input, such as a clinician sending a telemetry command to the IMD. In the MRI mode, features of the IMD are controlled via special programming to prevent or mitigate the above-noted issues caused by the MRI system. However, an error may occur in which the IMD is not placed into an MRI mode. For example, a technician may be unaware that the patient has an IMD and/or various MRI scanning facilities may not have programming equipment for switching the IMD to an MRI mode. As another example, the technician operating the MRI system (or other facility employee or volunteer) or a guardian of the patient having the MRI scan may have an IMD implanted therein, and that technician, guardian, etc. may become present within the threshold distance of the MRI system during the MRI scan, such that they experience electromagnetic fields above a threshold signal strength on their implanted IMD. In such examples, the identification of the presence-absence state of the MRI system is used as a safety feature in case of an error. In other examples, the IMD may not be designed for manually switching to the MRI mode and/or the one or more features may be controlled in response to sensing a presence-absence state of the MRI system, with or without the manual control. In various examples, the IMD may be placed or remain in a normal or default mode of operation in response to identifying an absence of the MRI system (e.g., an absent state).

FIGS. 2A-2B are block diagrams schematically illustrating an example IMD system 20. FIG. 2A illustrates the IMD system 20 in the presence of an MRI system 21. The IMD system 20 includes an IMD 22, at least one implantable sensor 25, an MRI engine 27, and an optional external device 26. Details on the various components are provided below.

In general terms, the IMD 22 is configured for implantation into a patient, and is configured to provide and/or assist in providing therapy to the patient. The at least one implantable sensor 25 may assume various forms, and is generally configured for implantation into the patient and to at least sense first data that is indicative of a presence-absence state of the MRI system 21. In various examples, the at least one implantable sensor 25 includes a sensor component in the form of or akin to a motion-based transducer. The motion-based transducer sensor component of the at least one implantable sensor 25 may be or include acceleration sensor such as an accelerometer (e.g., a multi-axis accelerometer such as a three-axis or six-axis accelerometer), a gyroscope, etc. In further examples, the at least one implantable sensor 25 includes more than one sensor, such as an acceleration sensor and non-acceleration sensor circuitry. The at least one implantable sensor 25 may be carried by the IMD 22, may be connected to the IMD 22, or may be a standalone component not physically connected to the IMD 22, as further described herein.

The MRI engine 27 is programmed to perform one or more operations as described below based upon data sensed via the at least one implantable sensor 25, such as an output of the at least one implantable sensor 25 being an input to the MRI engine 27. In general terms, the MRI engine 27 receives the first data from the at least one implantable sensor 25 and is programmed (or is connected to a separate engine that is programmed) to recognize, identify or detect a presence-absence state of the MRI system 21 based, at least in part, upon first data from the at least one implantable sensor 25. In some examples, the MRI engine 27 is programmed (or is connected to a separate engine that is programmed) to affect (or not effect) one or more features or the like relating to operation of the IMD system 20 in response the identified presence-absence state of the MRI system 21. The MRI engine 27 (or the algorithms as described below) may reside partially or entirely with the IMD 22, partially or entirely with the external device 26, or partially or entirely with a separate device or component (e.g., the cloud, etc.). Where provided, the external device 26 may wirelessly communicate with the IMD 22, and is operable to facilitate performance of one or more operations as described below. For example, the external device 26 may be used to initially program the IMD 22, and the IMD 22 then processes information (e.g., first data) and delivers care independent of the external device 26. In other examples, the external device 26 may be omitted. In such examples, the IMD 22, the at least one implantable sensor 25 and the MRI engine 27 perform one or more of the operations described below without the need for the external device 26 or human input. The MRI engine 27 may be further programmed to provide information to the patient and/or caregiver relating to the presence-absence state of the MRI system 21 and/or logged data during the presence-absence state of the MRI system 21 (e.g., in response to an identified presence of the MRI system 21) or other information of possible interest implicated by information from the at least one implantable sensor 25. In some examples, the MRI engine 27 may provide information indicating the presence-absence state of the MRI system 21 to another engine of the IMD 22 that is programmed to provide the information to the patient and/or caregiver relating to the presence-absence state of the MRI system 21 and/or the logged data.

The MRI engine 27 (or the logic akin to the MRI engine 27) may be incorporated into a distinct engine or engine programmed to perform certain tasks. For example, the logic of the MRI engine 27 as described below may be part of a care engine and utilized in controlling care provided to the patient, such as stimulation therapy delivered to the patient. Logic embodied by the MRI engine 27 may identify or detect the presence-absence state (e.g., a presence, a non-presence, an absence, and/or a non-absence) of the MRI system 21 in various manners. In some examples, the presence-absence state of the MRI system 21 may be recognized by a relatively straightforward algorithm that references only data from the at least one implantable sensor 25. As an example, if the first data from the at least one implantable sensor 25 includes a particular pattern, then the presence-absence state of the MRI system 21 is identified. In some examples, the presence-absence state of the MRI system 21 may be identified with reference to the data from the at least one implantable sensor 25 along with information from other data sources, such as data from a second sensor or a certain time (or range of times) of the day. An example second sensor includes a second implantable sensor carried by the IMD 22 such as an electromagnetic field sensor, heart rate monitor, respiration sensor, etc. In some examples, the presence-absence state of the MRI system 21 may be recognized with reference to data from the at least one implantable sensor 25, data from other data sources, and a data model (e.g., modeling or artificial intelligence or artificial learning). For example, one or more data sources (including data from the at least one implantable sensor 25) may be employed in a probabilistic decision model to recognize or identify a distinction between patterns indicative of a presence of the MRI system 21 and other activities and/or patterns indicative of an absence of the MRI system 21, among others.

With these and related examples, the MRI engine 27 is programmed to evaluate the probability of the presence-absence state of the MRI system 21 and/or that the IMD 22 is exposed to or will be exposed to electromagnetic fields 23 from an MRI scan, and deem or decide that the MRI system 21 is present (for purposes of initiating an operational control routine as described below) when the evaluated probability is acceptably high enough. As an example, the presence of the MRI system 21 may be recognized in response to a likelihood of occurrence being greater than a threshold, such as 80 percent or greater. Determining a probability may include weighting different factors and summing the weights to determine the probability. The factors may comprise, but are not limited to, the first data and the second data, such as the patient-volitional data and patient non-volitional data, as well as patterns identified within the first data, the second data and/or other inputs, such as a time of day. The factors may be weighted based on a relevancy of the factors (or relevancy of a value of the factor) to identifying a presence or an absence of an MRI system 21 (e.g., factor indicates MRI system likely present or not). As particular examples, a time of day or night may be weighted against the presence of the MRI system 21 (and/or weighted to indicate an absence of an MRI system 21) while particular body motion and/or posture patterns (e.g., patient in generally horizontal position, sliding motions, etc.), electromagnetic fields, and/or additional physiological parameters (e.g., low heart rate) may be weighted to indicate a presence of an MRI system 21. As further described herein, the probability may be revised over time based on additionally obtained data. Similarly, the absence of the MRI system 21 may be recognized in response to a likelihood of occurrence being greater than a threshold, such as 80 percent or greater. Although examples are not so limited and other thresholds may be used, such as 70 percent, 75 percent, 85 percent, or 90 percent. In various examples, identifying the presence-absence state of the MRI system 21 comprises identifying both the likelihood of the presence of the MRI system 21 and the likelihood of the absence of the MRI system 21, which may occur concurrently and/or at different times.

In some examples, when identifying a presence-absence state, the identification of the presence of the MRI system 21 may differ from the identification of the absence of the MRI system 21 at least because at least some of the particular sensing modalities for best identifying or determining each (presence verses absence) may be different and/or the particular value of sensed parameters may be different in presence of the MRI system 21 than in absence of the MRI system 21. In various examples, different threshold probabilities may be used to identify a presence of the MRI system 21 and to identify an absence of the MRI system 21. In some examples, a higher threshold may be used for identifying the presence of the MRI system 21 as compared to the absence of the MRI system 21, such that the IMD 22 may error on the side of normal-operations, as further described herein. In various examples, a higher threshold may be used for identifying the absence of the MRI system 21 as compared to the presence of the MRI system 21, such that the IMD 22 may error on side of protecting the IMD 22 from electromagnetic field effects. If both a presence and an absence of the MRI system 21 are identified, one may override the other, such as a presence overriding an absence or an absence overriding a presence identification.

Although the above examples describe identifying the presence-absence state of the MRI system 21 comprising identifying a likelihood of the presence of the MRI system 21 and/or identifying a likelihood of the absence of the MRI system 21, examples are not so limited. In some examples, at least some of the substantially same above-described features and attributes used to identify a presence-absence state may be used to identify a non-presence state and/or a non-absence state of the MRI system 21 relative to the IMD 22. In some such examples, the term “non-presence” may correspond to a probability of the presence of the MRI system 21 remaining below a presence threshold, while in some such examples, the term “non-absence” may correspond to a probability of absence of the MRI system 21 remaining below an absence threshold.

In some examples, the at least one implantable sensor 25 may include or be implemented as a wireless communication circuit which may wirelessly communicate with the MRI system 21 according to known wireless protocols, as further described herein. In such examples, the at least one implantable sensor 25 may detect the first data which includes a signal or other data message from a component of the MRI system 21, such as the external device 26. The MRI system 21 and/or the external device 26 which may be proximal to the MRI system 21 may include a beacon that emits a signal that is receivable by the IMD 22. For example, the wireless communication circuit of the IMD 22 detects the beacon-emitted signal (e.g., a data message) and provides the signal to the MRI engine 27 for processing. In some examples, in response to the signal, the MRI engine 27 of the IMD 22 may identify or detect the presence-absence state of the MRI system 21. In some examples, the MRI engine 27 and/or another component of the IMD 22 may respond to the signal by executing a security handshake protocol. In addition and/or alternatively, the beacon of the MRI system 21 and/or the external device 26 may notify an operator of the MRI system 21 that an MRI-sensitive device has entered the MRI zone and/or notify the patient of the situation, such as with an audible, visual and/or sensation alert (e.g., vibration and/or stimulation delivered to the patient) and/or a data message sent to another device.

In further examples, instead of and/or in addition to the MRI system 21 and/or the external device 26 providing the signal, the IMD 22 may include a beacon that emits a signal that is receivable by the MRI system 21 and/or other device. In such examples, the MRI system 21 may respond to the beacon by providing an alert and/or a data message to notify the operator of the MRI system 21 and/or the patient.

FIG. 2B illustrates the IMD system 20 in the presence of a pseudo-MRI system 24. Additional types of devices and/or medical equipment, other than an MRI system, may exert electromagnetic fields 23 above a threshold signal strength which may impact the IMD 22. Such devices and/or equipment are herein generally referred to as “pseudo-MRI systems”. In various examples, the at least one implantable sensor 25 may sense first data that is indicative of a presence-absence state of the pseudo-MRI system 24. The MRI engine 27 of the IMD system 20 may identify a presence-absence state of the pseudo-MRI system 24 relative to the IMD 22 based, at least in part, upon the first data from the at least one implantable sensor 25. In this manner, the MRI engine 27 may function as (or alternatively comprises) a pseudo-MRI engine. The operations of the at least one implantable sensor 25 and the MRI engine 27 of FIG. 2B may substantially include the same features and/or operations of the least one implantable sensor 25 and the MRI engine 27 (including the identification of the presence-absence state of the MRI system 21 and/or adjusting a feature of IMD 22 in response) as described in FIG. 2A and as further described herein, but with the presence-absence state being with respect to the pseudo-MRI system 24. Although examples are not limited to MRI systems, the following examples generally refer to MRI systems for ease of reference.

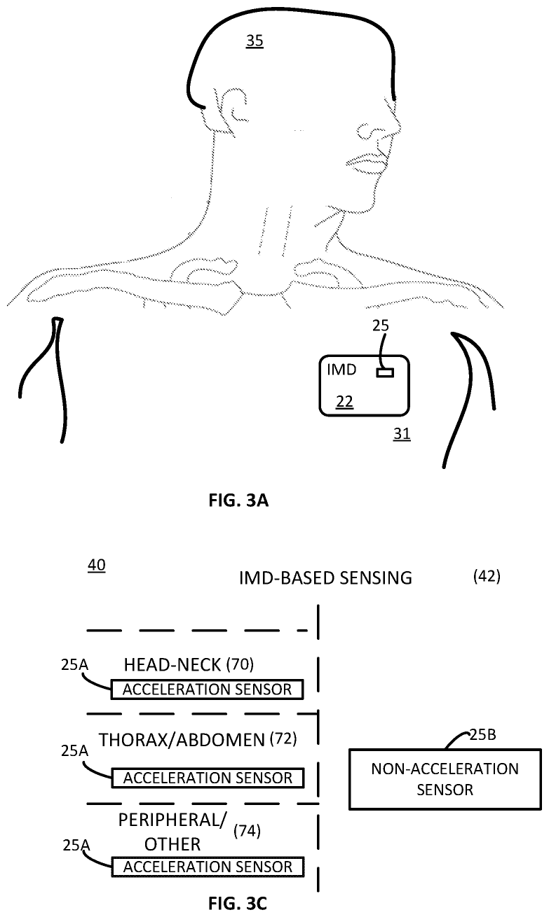

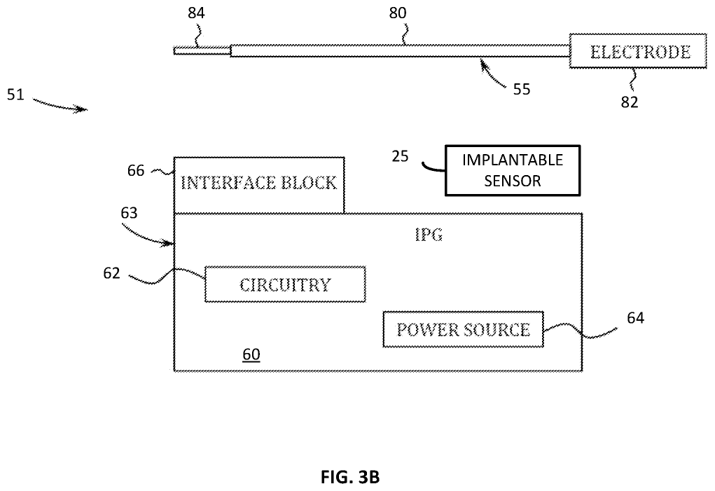

FIGS. 3A-3C are diagrams schematically representing deployment of an example IMD, which includes an implantable sensor arrangement. More specifically, FIG. 3A is diagram including a front view schematically representing deployment of an example IMD 22, which includes at least one implantable sensor 25. As shown in FIG. 3A, in some examples the IMD 22 (and therefore the at least one implantable sensor 25) may be chronically implanted in a pectoral region 31 of a patient 35. The at least one implantable sensor 25 may comprise an acceleration sensor that senses first data including various physiologic phenomenon sensed from this implanted position (e.g., body motion, posture, vibrations, such as anatomy vibrations and device vibrations).

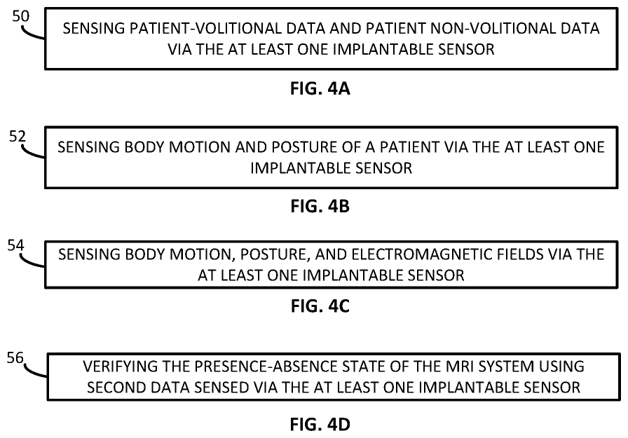

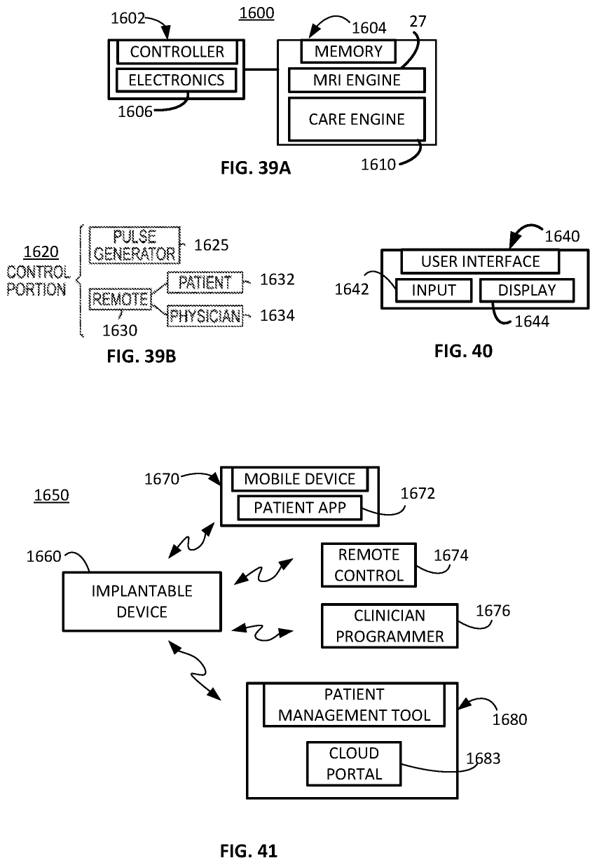

As noted above, the first data sensed via the at least one implantable sensor 25 may comprise patient-volitional data and patient non-volitional data from which, a presence-absence state of the MRI system may be identified. Sensing the patient-volitional data and patient non-volitional data is further described below in association with at least FIGS. 4A-4D. In some examples, the IMD 22 may comprise an implantable pulse generator (IPG), such as for managing sensing and/or stimulation therapy, as later described in association with at least FIGS. 39-41.

FIG. 3B is a block diagram schematically representing one example of an IMD 51 which is an example implementation of, and/or may comprise at least substantially the same features and attributes of IMD 22 of the IMD system 20 of FIGS. 2A-2B. The IMD 51 may include an IPG assembly 63 and at least one stimulation lead 55. The IPG assembly 63 may include a housing 60 containing circuitry 62 and a power source 64 (e.g., battery), and an interface block or header-connector 66 carried or formed by the housing 60. The housing 60 is configured to render the IPG assembly 63 appropriate for implantation into a human body, and may incorporate biocompatible materials and hermetic seal(s). The circuitry 62 may include circuitry components and wiring appropriate for generating desired stimulation signals (e.g., converting energy provided by the power source 64 into a desired stimulation signal), for example in the form of a stimulation engine. In some examples, the circuitry 62 may include telemetry components for communication with external devices. For example, the circuitry 62 may include a transmitter that transforms electrical power into a signal associated with transmitted data packets, a receiver that transforms a signal into electrical power, a combination transmitter/receiver (or transceiver), an antenna (e.g., an inductive telemetry antenna), etc.

In some examples, the stimulation lead 55 includes a lead body 80 with a distally located stimulation electrode 82. At an opposite end of the lead body 80, the stimulation lead 55 includes a proximally located plug-in connector 84 which is configured to be removably connectable to the interface block 66. For example, the interface block 66 may include or provide a stimulation port sized and shaped to receive the plug-in connector 84.

In general terms, the stimulation electrode 82 may optionally be a cuff electrode, and may include some non-conductive structures biased to (or otherwise configurable to) releasable secure the stimulation electrode 82 about a target nerve. Other formats are also acceptable. Moreover, the stimulation electrode 82 may include an array of contact electrode to deliver a stimulation signal to a target nerve. In some non-limiting examples, the stimulation electrode 82 may comprise at least some of substantially the same features and attributes as described within at least: U.S. Pat. No. 8,340,785, issued Dec. 25, 2012, and entitled “SELF EXPANDING ELECTRODE CUFF”; U.S. Pat. No. 9,227,053, issued Jan. 5, 2016, and entitled “SELF EXPANDING ELECTRODE CUFF”; U.S. Pat. No. 8,934,992, issued Jan. 13, 2015, and entitled “NERVE CUFF”; and/or U.S. Patent Publication No. 2020/0230412, published on Jul. 23, 2020, and entitled “CUFF ELECTRODE”, the entire teachings of each of which are incorporated herein by reference in their entireties. Examples are not limited to cuffs and may include stimulation elements having a stimulation electrode 82 in different types of arrangements and/or for different targets, such as an Ansa cervicalis (AC) target, a paddle, and an axial arrangement, among others.

In some examples, the lead body 80 is a generally flexible elongate member having sufficient resilience to enable advancing and maneuvering the lead body 80 subcutaneously to place the stimulation electrode 82 at a desired location adjacent a nerve, such as an airway-patency-related nerve (e.g., hypoglossal nerve, phrenic nerve, ansa cervicalis nerve, etc.). In some examples, such as in the case of obstructive sleep apnea, the nerves may include (but are not limited to) the nerve and associated muscles responsible for causing movement of the tongue and related musculature to restore airway patency. In some examples, the nerves may include (but are not limited to) the hypoglossal nerve and the muscles may include (but are not limited to) the genioglossus muscle. In some examples, lead body 80 may have a length sufficient to extend from the IPG assembly 63 implanted in one body location (e.g., pectoral) and to the target stimulation location (e.g., head, neck). Upon generation via the circuitry 62, a stimulation signal is selectively transmitted to the interface block 66 for delivery via the stimulation lead 55 to such nerves.

The at least one implantable sensor 25 may be connected to the IMD 51 in various fashions. For example, the at least one implantable sensor 25 may include a lead body carrying the motion-based transducer sensor element of an acceleration sensor at a distal end, and a plug-in connector at proximal end. The plug-in connector may be connected to the interface block 66, such as the interface block 66 including or providing a sense port sized and shaped to receive the plug-in connector of the at least one implantable sensor 25, and the lead body extended from the IPG assembly 63 to locate the sensor element at a desired anatomical location. Alternatively, the at least one implantable sensor 25 may be physically coupled to the interface block 66, and thus carried by the IPG assembly 63. In such examples, the at least one implantable sensor 25 may be considered a component of the IMD 51. In some examples the physical coupling of the at least one implantable sensor 25 relative to the IPG assembly 63 is performed prior to implantation of those components.

In some examples, the at least one implantable sensor 25 (and in particular, at least the motion-based transducer sensor component as described above) may be incorporated into a structure of the interface block 66, into a structure of the housing 60, and/or into a structure of the stimulation lead 55. With these and similar configurations, the sensor component of the at least one implantable sensor 25 is electronically connected to the circuitry 62 within the housing 60 or other enclosure of the IPG assembly 63. More specifically, the at least one implantable sensor 25 may be connected in various orientations as described within U.S. patent application Ser. No. 16/978,275, filed on Sep. 4, 2020, and entitled “SYSTEMS AND METHODS FOR OPERATING AN IMPLANTABLE MEDICAL DEVICE BASED UPON SENSED POSTURE INFORMATION”, the entire teachings of which is incorporated herein by reference in its entirety. Although the above examples describe an IMD 51 having a stimulation lead 55, examples are not so limited and example IMDs may additionally or alternatively include a lead used for sensing, such as a lead used to sense for the presence-absence state of an MRI system and/or a lead used for sensing data that is unrelated to an MRI system.

In some examples, the at least one implantable sensor 25 may be wirelessly connected to the IMD 51. In such examples, the interface block 66 need not provide a sense port for the at least one implantable sensor 25 or the sense port may be used for a second sensor (not shown). In some examples, the circuitry 62 of the IPG assembly 63 and circuitry (not shown) of the at least one implantable sensor 25 communicate via a wireless communication pathway according to known wireless protocols, such as Bluetooth, near-field communication (NFC), Medical Implant Communication Service (MICS), 802.11, etc. with each of the circuitry 62 and the at least one implantable sensor 25 including corresponding components for implementing the wireless communication pathway. In some examples, a similar wireless pathway is implemented to communicate with devices external to the patient's body for at least partially controlling the at least one implantable sensor 25 and/or the IPG assembly 63, to communicate with other devices (e.g., other sensors) internally within the patient's body, or to communicate with other sensors external to the patient's body.

FIG. 3C is a diagram 40 schematically representing example IMD-sensing arrangement 42 that includes an acceleration sensor 25A and non-acceleration sensor circuitry 25B deployed relative to a patient's body. As shown in FIG. 3C, in some examples an acceleration sensor 25A may be implanted internally to sense patient-volitional data such as in a head-and-neck region 70, a thorax/abdomen region 72, and/or a peripheral/other region 74. Although not illustrated, the acceleration sensor 25A may sense patient non-volitional data, such as vibrations that are induced by an external source. The vibration may include anatomical vibrations and/or device vibrations sensed by the acceleration sensor 25A. In some examples, more than one acceleration sensor 25A may be implanted in a single region and/or in different multiple regions in the patient's body. As further shown in FIG. 3C, in some examples non-acceleration sensor circuitry 25B (like 25A) may be deployed internal to a patient's body and is used to sense patient non-volitional data and/or patient-volitional data, such as additional physiological data. Some examples of non-acceleration sensor circuitry 25B are further illustrated at least by FIGS. 5A-10.

FIGS. 4A-4D are block diagrams, which may comprise part of a flow diagram in an example method (e.g., method 10). As shown at 50 in FIG. 4A, sensing the first data may comprise sensing patient-volitional data and patient non-volitional data via the at least one implantable sensor. For example, as shown at 52 in FIG. 4B, sensing the first data (or second data) comprises sensing body motion and posture of a patient via the at least one implantable sensor. In some examples, as shown at 54 in FIG. 4C, sensing the first data comprises sensing body motion and posture of the patient, as well as electromagnetic fields via the at least one implantable sensor. The first data may be sensed by an acceleration sensor and the method may include, as shown at 56 in FIG. 4D, verifying the presence-absence state of the MRI system, such as the presence of the MRI system, using second data sensed by the at least one implantable sensor. As used herein, in some examples the first data comprises and/or refers to data sensed via the at least one implantable sensor of the IMD system used to initially identify a presence-absence state of the MRI system. In some examples, the second data comprises and/or refers to data sensed subsequent to the first data via the at least one implantable sensor of the IMD system. For example, the second data may be sensed later in time from the first data. In some examples, the second data is used to verify the presence-absence state of the MRI system initially identified via the first data. In some examples, the second data may be used to override the presence-absence state of the MRI system identified via the first data.

One such example of sensing second data (e.g., later in time than sensing the first data) comprises sensing second data at completion of the MRI scan to determine that the MRI scan is no longer exerting an MRI field and/or to determine whether the patient has left the vicinity of the MRI system The first data and/or second data may include vibrations sensed by the acceleration sensor, which are indicative of electromagnetic fields from an MRI scan and/or electromagnetic fields sensed via non-acceleration sensor circuitry, such as an MRI-sensitive conductive element, Hall effect sensor, magnetometer, etc. In some such examples, the first data and/or second data may be determined using the at least one implantable sensor according to at least the examples described in association with FIGS. 5A-9.

The various methods illustrated herein, such as method 10 described in associated with FIG. 1 and/or FIGS. 4A-4D, may be implemented by the IMD systems and/or IMDs illustrated herein, such as by the IMD system 20 of FIG. 2. For example, the IMD system 20 of FIG. 2 may perform the various actions of the methods described herein.

FIGS. 5A-5F are block diagrams schematically illustrating example IMDs. As shown by FIG. 5A, an example IMD 100 includes an acceleration sensor 110 and an MRI-sensitive conductive element 115 as a first implantable sensor and a second implantable sensor.

The acceleration sensor 110 may comprise an accelerometer (e.g., a single axis or multi-axis accelerometer), a gyroscope, a pressure sensor, etc. The acceleration sensor 110 may provide information along a single axis, or along multiples axes (e.g., three-axis accelerometer, three-axis gyroscope (three rotational axes), six-axis accelerometer (three linear axes and three rotational axes), nine-axis accelerometer (three linear axes, three rotational axes and three magnetic axes), etc. Regardless of an exact form, the sensor component of the acceleration sensor 110 is capable of sensing, amongst other things, information indicative of body motion of the patient, a posture of the patient, and vibrations induced by external sources (e.g., the MRI system). As a point of reference, while information generated by the acceleration sensor 110 is signaled to and acted upon by the IMD 100 (such as by an MRI engine 27 of an IMD 22 of FIG. 2A), information from the acceleration sensor 110 may be utilized by other modules or engines, such as by a care engine that manages care or diagnostic data provided to the patient by the IMD as described below. In some non-limiting examples, the acceleration sensor 110 may form part of the IMD 100 or is otherwise coupled to the IMD 100, as previously described.

The following provides some examples of sensing information indicative of body motion, posture, and vibrations by the acceleration sensor 110, however examples are not so limited and the acceleration sensor 110 may sense body motion, posture, and vibration using a variety of techniques. The acceleration sensor 110 may be used to generate the first data via sensing of forces in multiple directions or axes. The acceleration forces may be indicative of body motion, posture of the patient, and/or vibrations caused by external sources, such as electromagnetic fields from an MRI scan. In some examples, the acceleration sensor 110 is a three-axis accelerometer that may sense or measure the static and/or dynamic forces of acceleration on three axes. Static forces include the constant force of gravity. By measuring the amount of static acceleration due to gravity, an accelerometer may be used to identify the angle it is tilted at with respect to the earth. By sensing the amount of dynamic acceleration, the accelerometer may find out how fast and in what direction the IMD is moving, which may be indicative of body movement. Single-and multi-axis models of accelerometers detect magnitude and direction of acceleration (or proper acceleration) as a vector quantity. With these and similar types of sensor constructions, an output from the acceleration sensor 110 may include vector quantities in one, two or three axes. For example, FIG. 5B provides an axis orientation indicator 121 of a three-axis accelerometer useful as the acceleration sensor 110 in some non-limiting examples. The three axes and three outputs of the three-axis accelerometer are conventionally labeled as X, Y, and Z, with the three axes X, Y, Z being orthogonal to one other.

In some examples, with these and related constructions, efforts may be made to implant the acceleration sensor 110 within the patient's body such that the axes X, Y, Z are in general alignment with planes or axes of the patient. For example, in FIG. 5C, a patient's body 202 may be viewed as having a left side 204 and an opposite right side 206, along with an anterior portion 208 and an opposite posterior portion 210. A conventional coordinate system of the patient's body 202 includes an anterior-posterior (A-P) axis and a lateral-medial (L-M) axis as labeled in FIG. 5C, and a superior-inferior (S-I) axis (vertical or head-to-toe) that is into a plane of the view of FIG. 5C. With the non-limiting example of FIG. 5C in which the acceleration sensor 110 is a three-axis accelerometer disposed within a housing of the IMD 100, the acceleration sensor 110 is arranged relative to the housing and relative to the patient's body 202 such that the sensor's X, Y, Z axes are approximately aligned with the patient's body coordinate system. For example, the Z axis of the acceleration sensor 110 may be aligned with A-P axis, the X axis aligned with the L-M axis, and the Y axis aligned with the S-I axis. A posture (including position) of the patient may be designated with reference to the body coordinate system, such that X, Y, Z information from acceleration sensor 110 may be employed to determine posture when the sensor axes X, Y, Z are aligned with the body coordinate system axes.

In some examples, exact alignment may be difficult to achieve. Similar concerns may arise where the acceleration sensor 110 is implanted at a location apart from the housing of the IMD 100. In various examples, some methods of the present disclosure may include calibrating data signaled from the acceleration sensor 110 for possible misalignment with the body coordinate system axes or other concerns relating to determining or designating a posture of the patient based on data from the acceleration sensor 110 as described within U.S. patent application Ser. No. 16/978,275, filed Sep. 4, 2020, and entitled “SYSTEMS AND METHODS FOR OPERATING AN IMPLANTABLE MEDICAL DEVICE BASED UPON SENSED POSTURE INFORMATION”, the entire teachings of which is incorporated herein by reference in its entirety.

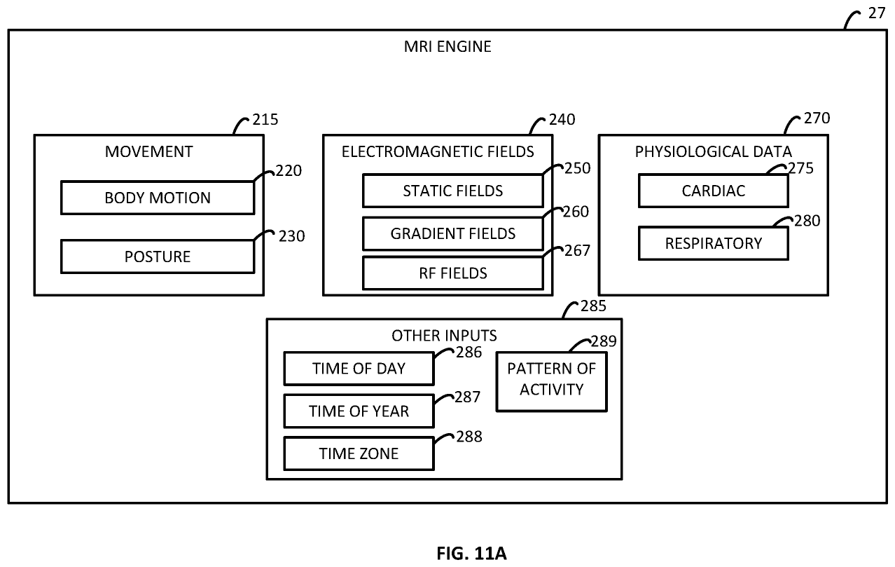

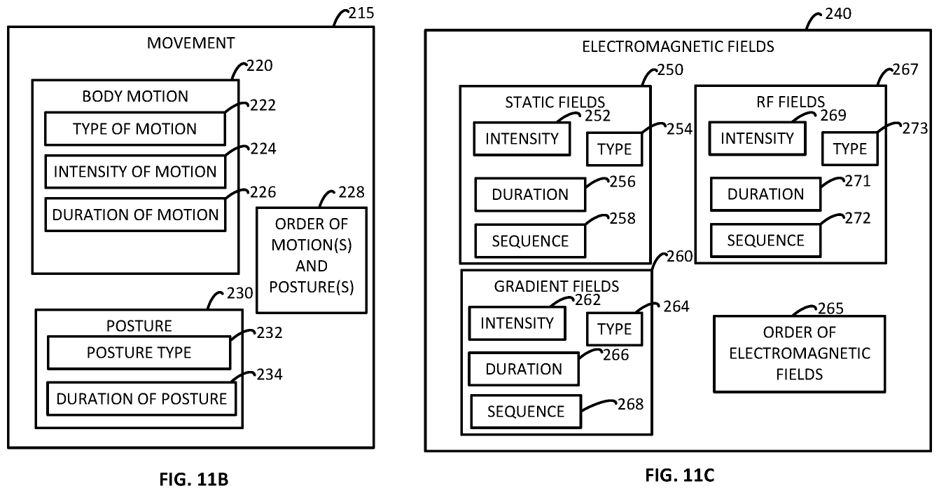

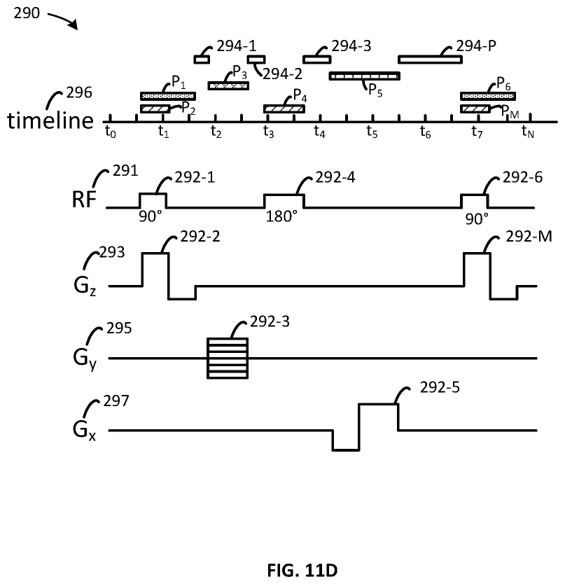

As noted above, sensing the amount of dynamic acceleration may be used to identify body motion and posture. Example body motions include movement in a vector or a direction (e.g., walking, running, biking), rotational motions (e.g., twisting), sliding motions (which may be caused by external sources), and changes in posture (e.g., change from an upright position to a sitting or supine position), among other movements. The motion may be sensed relative to a gravity vector, such as an earth gravity vector and/or a vertical baseline gravity vector for calibrating the data. In various examples, the sensed force(s) may be processed to determine a posture of the patient. As used herein, posture refers to or includes a position or bearing of the body. In some instances, the term “posture” may sometimes be referred to as “body position”. Example postures include upright or standing position, supine position (e.g., generally horizontal body position), a generally supine reclined position, sitting position, etc. Further detail on examples of identifying or determining motion and posture are described below in connection with the example MRI engine 27 of an IMD and sub-engines illustrated by FIGS. 11A-11C.

The acceleration sensor 110 may sense vibrations caused by the electromagnetic fields exerted by an MRI system during an MRI scan and which the IMD 100 is exposed to. In particular, the electromagnetic fields exerted by the MRI system may generate relatively loud noises and vibrations. The vibrations caused by the MRI system may be due to an interaction between the gradient induced eddy current magnetic moment and the MR scanner static magnetic field. In other examples and/or in addition, the vibrations may be caused by or based on low level quantities of ferrite material in the IMD 100. The vibrations may be in a series of repeated step functions, which may be used to distinguish vibrations caused by an MRI scan from other vibrations for everyday activities which exhibit sinusoidal vibration patterns. For example, the electromagnetic fields of the MRI scan may cause vibrations in a pattern, such as no vibrations (e.g., off), step functions, no vibrations, step functions, and which may be repeated. Even with changes in the duration, frequency, and/or magnitude of the vibrations from different MRI systems, the underlying pattern of the vibrations may have the same characteristics of the series of repeated step functions.

More specifically, the time-varying gradient magnetic fields exerted by MRI systems, as further illustrated by FIG. 11D, include a combination of Gx, Gy and Gz waveforms. The signals sensed by the acceleration sensor 110 may be indicative of vibrations caused by the Gx, Gy and Gz waveforms, as well as the RF pulses, overtime. An example vibration pattern may be indicative of RF pulses followed by changes in gradient magnetic fields, the pattern of which is repeated a number of times and may generate periodic burst phenomenon. In some examples, an MRI engine may comprise or have access to a plurality of stored vibration patterns which may be used to identify a matching pattern indicative of the presence-absence state of an MRI system and to identify the presence-absence state comprises a presence of the MRI system. In some examples, at least one threshold may be used to identify the electromagnetic fields, with the thresholds optionally being used after an identified pattern of motion and posture indicative of the patient sitting on the tray of the MRI system, as further described herein. As an example, a threshold vibration and/or threshold electrical signal (e.g., voltage) associated with vibrations caused by gradient magnetic fields and RF pulses may be used to identify the electromagnetic fields.

With further reference to FIG. 5A, the vibration data sensed by the acceleration sensor 110 may be processed, such as by the MRI engine (e.g., MRI engine 27 of FIG. 2A) or other engine of the IMD 100 or in communication with the IMD 100, to detect the electromagnetic fields exerted by the MRI system (e.g., detect RF fields, static magnetic fields and/or time-varying gradient magnetic fields). For example, based on the vibration pattern identified, the electromagnetic fields of the MRI system are identified. The IMD 100 may analyze the resulting sensor outputs (e.g., vibration data sensed by the acceleration sensor 110) by performing fast Fourier transform (FFT), wavelet transform, or other data processing. The IMD 100, via other circuitry 112, may monitor one or more vibrational characteristics (e.g., amplitude, frequency, and/or duty cycle) of mechanical vibrations sensed by the acceleration sensor 110 when exposed to a magnetic gradient field generated by the MRI system.

In some examples, the IMD 100 detects, over time, the vibrational characteristics associated with the vibration data. As the magnetic gradient field is exerted in a pattern, which may be achieved by varying a magnitude, frequency, and/or duty cycle of the magnetic gradient field, a variance in the magnetic gradient field corresponds to a variance in the vibrations sensed by the acceleration sensor 110. For example, as noted above, the frequency of the RF pulses (e.g., MHz range), which is dependent of the magnitude of the static magnetic fields, may be different than the frequency of the gradient magnetic fields (e.g., kHz range) and may cause different vibrational characteristics as sensed by the acceleration sensor 110.

As an example, an FFT is performed on one or more sensor signals, or a composite of multiple sensor signals, produced by the acceleration sensor 110. The results of the FFT are used to determine whether the IMD 100 is being exposed to a time-varying gradient magnetic field from an MRI system. In some examples, the results of an FFT may be compared to known vibration patterns of time-varying gradient magnetic field (and optionally RF pulses) sequences produced by example MRI systems to determine whether the IMD is being exposed to time-varying gradient magnetic field produced by an MRI system. Alternatively, or additionally, the magnitude of the results of an FFT may be compared to a corresponding threshold, to determine whether the IMD is being exposed to a time-varying gradient magnetic field from an MRI system. The threshold may be a threshold vibration (or vibration characteristic, such as a frequency) and/or a threshold electrical signal associated with a vibration caused by gradient magnetic fields and/or RF pulses of example MRI systems. In some instances, multiple thresholds may be used to distinguish and/or identify both gradient magnetic fields and RF pulses exerted by an MRI system, such as a first threshold associated with vibration caused by gradient magnetic fields (e.g., frequency in the kHz range) and a second threshold associated with vibration caused by RF pulses (e.g., frequency in the MHz range, such as 6.4 to 128 MHz), although examples are not so limited and may include identifying the vibration pattern using known patterns and without the use of thresholds.

In various examples, the acceleration sensor 110 may be used to sense additional physiological data. The additional physiological data may include additional physiological parameters, such as cardiac signals and/or respiration information. As further described herein, the respiration information may be determined based on rotational movements of a portion of a chest wall of the patient during breathing. For example, the acceleration sensor 110 may be used to determine respiration information based on rotational movements of a chest wall of the patient as described within U.S. patent application Ser. No. 16/977,664, filed on Sep. 2, 2020, and entitled “RESPIRATION DETECTION”, the entire teachings of which is incorporated herein by reference in its entirety.

The MRI-sensitive conductive element 115 may be used to detect electromagnetic fields exerted by the MRI system and/or distinguish between two or more of RF fields, gradient magnetic fields and static magnetic fields. In some examples, an MRI-sensitive conductive element, as used herein, includes a conductive structure or material used to sense electromagnetic fields. Examples of an MRI-sensitive conductive element include a conductive wire, a conductive loop or coil, an antenna, a lead, and other internal circuit components of the IMD 100, among other types of conductive elements. In some examples, the MRI-sensitive conductive element 115 may include an inductive telemetry antenna, such as a coil with or without a ferrite element, which may generally and/or normally be used for communication and is further used to detect static magnetic fields (e.g., during patient movement) and/or gradient magnetic fields (e.g., when patient is motionless or moving). In some examples, the MRI-sensitive conductive element 115 may include a power supply inductor, such as a coil with or without a ferrite element, which may generally and/or normally be used for switching power supply (e.g., voltage conversion) and is further used to detect static and/or gradient magnetic fields. The electromagnetic fields exerted by the MRI system may cause a voltage on the MRI-sensitive conductive element 115 which may be detected via an electrical signal. The electrical signal and/or a pattern of electrical signals may be indicative of the RF fields, static magnetic fields and gradient magnetic fields exerted by an MRI system. Other circuitry 112 may detect the electrical signal(s) on the MRI-sensitive conductive element 115, such as a comparator, an amplifier and/or processing circuitry. The RF fields, static magnetic fields and gradient magnetic fields may be associated with different electrical signal thresholds, such as a first signal threshold associated with a minimum value of a static magnetic field exerted by example MRI systems, a second signal threshold associated with a minimum rate of change of a gradient magnetic field, i.e., dB/dt, and/or a slew rate of example MRI systems, and/or a third signal threshold associated with RF pulses exerted by example MRI systems (e.g., associated with an amplitude and/or frequency of the RF pulses). The gradient magnetic fields may vary over time and exhibit a particular pattern (with the RF fields) such as the rate of change and/or a slew rate of the electromagnetic field strength which cause the induced electrical signal(s), such as voltage(s), on the MRI-sensitive conductive element 115 that is greater than the second signal threshold.

As noted above, example MRI systems may exert a static magnetic field that is greater than (e.g., is large) 0.2-3.0 Tesla (T), which may be exerted on the patient and the IMD 100 when the patient is moved into the bore of the scanner. The motion of the IMD 100 moving through the static field causes a voltage signal over the first signal threshold to be induced on the MRI-sensitive conductive element 115. In other examples or in addition, the IMD 100 may detect a change in the voltage signal on the MRI-sensitive conductive element 115 (which is greater than a threshold change). Example MRI systems may exert gradient magnetic fields at greater than a minimum rate of change (dB/dt), wherein d is delta, B is the gradient magnetic fields, and t is time, and may exhibit a particular slew rate (e.g., a maximum gradient strength of the gradient divided by the rise time). In some examples, the first voltage threshold may be associated with a voltage induced on the MRI-sensitive conductive element 115 due to electromagnetic fields greater than 0.2-3.0 T and the second signal threshold may be associated with a voltage or changes in voltages induced on the MRI-sensitive conductive element due to a slew rate in the gradient magnetic fields of 25-400 millitesla per meter per microsecond (mT/m/ms) (or 50-200 T per meter per second). The first and second signal thresholds may be different for different types of MRI-sensitive conductive elements. Although the above examples describe detecting a voltage, examples are not so limited and may include any electrical signal, for example, impedance measurements, current measurements, and resistance measurements, among others. Accordingly, the above-described examples of signal thresholds encompass voltage thresholds, current thresholds, impedance thresholds, and various other electrical signals thresholds. Further, examples may include a third signal threshold associated with RF pulses, as described above. Additionally, examples are not limited to use of signal thresholds, and may comprise identifying an electrical signal pattern, such as a voltage pattern, using known patterns of example MRI systems and without the use of thresholds.

With continued reference to FIG. 5A, the IMD 100 may further comprise a magnetometer 111 to sense electromagnetic fields. The electromagnetic fields may be distinguished using a first Tesla (T) threshold that is indicative of a magnitude of static magnetic fields exerted by example MRI systems (e.g., first threshold of 0.2-3.0 T), a second T threshold that is indicative of gradient magnetic fields, such as a time derivative of the gradient magnetic field (e.g., a second threshold or a slew rate of 25-400 mT/m/ms), and a third T threshold that is indicative of RF pulses, such as T threshold associated with a frequency and/or amplitude of the RF pulses. More specifically, the magnetometer 111 may be used to detect static magnetic fields from an MRI system based on the first T threshold (e.g., 0.2-3.0 T) and gradient magnetic fields based on the second T threshold (e.g., 25-400 mT/m/ms). Similarly to the above, examples are not limited to use of electromagnetic field thresholds, and may comprise identifying an electromagnetic field pattern using known patterns of example MRI systems, thresholds based on the electric signal(s) generated by the magnetometer 111, and/or without the use of thresholds.

In various examples, the data or signal sensed by the acceleration sensor 110, by the MRI-sensitive conductive element 115, and/or the magnetometer 111 may be used in combination to identify the presence-absence state of the MRI system. For example, the vibrations sensed by the acceleration sensor 110 may be used in combination with electrical signals (e.g., voltages, current) induced on the MRI-sensitive conductive element 115 and/or the electromagnetic fields sensed by the magnetometer 111 to verify the presence-absence state of the MRI system, such as detecting RF fields, gradient magnetic fields and/or static magnetic fields based on the pattern of vibrations, the pattern of electrical signals, and the pattern of electromagnetic fields. In other examples and/or in addition, body motion and posture sensed via the acceleration sensor 110 may be used in combination with electrical signals induced on the MRI-sensitive conductive element 115 and/or the electromagnetic fields sensed by the magnetometer 111 to verify the presence-absence state of the MRI system. As an example, the above described vibration threshold(s), signal thresholds (e.g., first voltage threshold and second voltage threshold), and/or electromagnetic field thresholds (e.g., the first T threshold and second T threshold) may be used in different combinations to detect electromagnetic fields using the acceleration sensor 110, the MRI-sensitive conductive element 115 and/or the magnetometer 111. As another example, body motion and posture data sensed by the acceleration sensor 110 may be used in combination with electrical signal data (e.g., voltage data) sensed via the MRI-sensitive conductive element 115 to identify a pattern of body motion, posture, and electrical signals that is indicative of the presence-absence state of the MRI system.

An example pattern may comprise a sliding body motion that occurs while the patient is in a generally horizontal position and while a voltage is induced on the MRI-sensitive conductive element 115 that is above a (first) signal threshold. Such a pattern may be indicative of (a likelihood of) the patient being moved into the bore of the scanner, which results in exertion of the MRI static magnetic field on the IMD 100. In such an example, second data sensed via the acceleration sensor 110 and/or the MRI-sensitive conductive element 115 may be used to verify the presence-absence state of the MRI system, such as verifying an identified presence of the MRI system which may be identified prior to performance of the MRI scan. The first data may be used to predict exposure of the IMD to RF pulses and/or gradient magnetic fields by the MRI system from an MRI scan, and the second data may be used to verify the prediction.

In some examples, identifying the presence-absence state of the MRI system may comprise identifying correlation of signals from two or more implantable sensors. For example, signals indicative of body motion (e.g., a sliding body motion while the patient is in a generally horizontal position from MRI table movement) sensed by the acceleration sensor 110 may be detected and correlated with an electrical signal induced on the MRI-sensitive conductive element 115 and/or the magnetometer 111 (e.g., from movement through the static field).

As shown by FIGS. 5D-5F, examples are not limited to IMDs having each of the acceleration sensor 110, the MRI-sensitive conductive element 115, and/or the magnetometer 111. As shown by FIG. 5D, an example IMD 101 may comprise an acceleration sensor 110 and the MRI-sensitive conductive element 115, and not the magnetometer 111. Another example IMD 102, as shown by FIG. 5E, may comprise an acceleration sensor 110 and magnetometer 111, and not the MRI-sensitive conductive element 115. A further example IMD 103, as shown by FIG. 5F, may comprise the MRI-sensitive conductive element 115 and the magnetometer 111, and not the acceleration sensor 110.

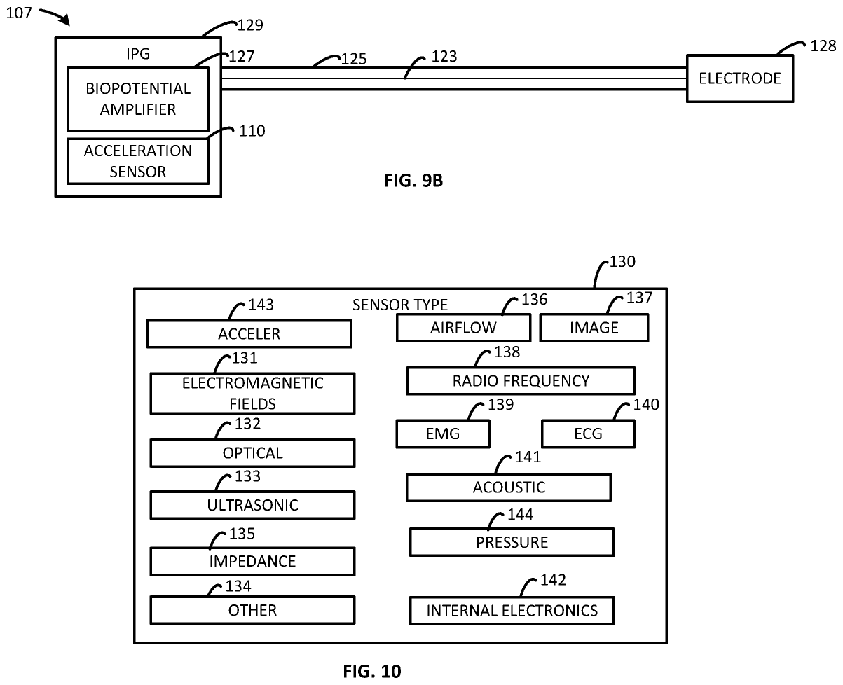

FIGS. 6-9B illustrate other example IMDs with implantable sensor combinations. As shown by FIG. 6, an example IMD 104 includes implantable sensors comprising the acceleration sensor 110 and the MRI-sensitive conductive element 115, as previously described, and a Hall effect sensor 117 which senses the electromagnetic fields. The example IMD 105, as shown by FIG. 7, includes the acceleration sensor 110, the MRI-sensitive conductive element 115, and a giant magnetoresistance sensor 120 which senses the electromagnetic fields. For example, a giant magnetoresistance sensor may detect electromagnetic fields by detecting changes in an electro resistance characteristic of the sensor. As shown by FIG. 8, the example IMD 106 includes the acceleration sensor 110, the MRI-sensitive conductive element 115, and a reed switch 122 which is used to sense the electromagnetic fields. And, the IMD 107 as shown by FIG. 9A includes the acceleration sensor 110, the MRI-sensitive conductive element that includes a lead 125, and a biopotential amplifier 127. The biopotential amplifier 127, which may be used by the IMD 107 to detect the additional physiological data (e.g., ECG, EKG, EMG, ENG), is repurposed as an electromagnetic field detector.

FIG. 9B illustrates an example IMD 107 which may be an implementation of and/or comprise substantially the same features as the IMD 107 illustrated by FIG. 9A. The IMD 107 illustrated in FIG. 9B comprises an IPG 129 that includes a biopotential amplifier 127 and an acceleration sensor 110, as described in connection with FIG. 9A, and the lead 125 is coupled to the IPG 129. The IPG 129 and the lead 125 may comprise at least some of substantially the same features and operations as the IPG 63 and lead 55 of FIG. 3B. A conductive loop is formed by the lead wire 123 to the lead electrode 128 of the lead 125, tissue of the patient and back to the housing (e.g., a conductive case) of the IPG 129 of the IMD 107 through the tissue. This forms a single turn antenna with a loop area. The electrical signal (e.g., voltage) induced on the lead 125 is proportional to the loop area and may be measured by the biopotential amplifier 127. As may be appreciated, examples are not limited to the implantable sensors and/or combinations as illustrated by FIGS. 5A-9, and may include a variety of different implantable sensors, combinations, and other circuitry, such as the other circuitry 112 illustrated by FIG. 5A and further described herein.

Although the examples illustrated by FIGS. 5A-9 show the at least one implantable sensor forming part of the IMD, examples are not so limited and one or more of the implantable sensors may be separate from the respective IMD.

FIG. 10 is a block diagram schematically representing an example sensor type 130. In some examples, sensor type 130 corresponds to a sensor (e.g., 25A in FIG. 3A) and/or a sensing function. As shown in FIG. 10, sensor type 130 comprises various types of sensor modalities 131-144, any one of which may be used for determining, obtaining, and/or identifying the presence-absence state of an MRI system, respiratory information, cardiac information, sleep quality information, sleep disordered breathing-related information, and/or other information related to providing patient therapy.

As shown in FIG. 10, in some examples sensor type 130 comprises the modalities of pressure 144, impedance 135, acceleration 143, electromagnetic field sensor 131 airflow 136, radio frequency (RF) 138, optical 132, electromyography (EMG) 139, electrocardiography (ECG) 140, ultrasonic 133, acoustic 141, image 137, internal electronics 142 and/or other 134. In some examples, sensor type 130 comprises a combination of at least some of the various sensor modalities 131-144.

It will be understood that, depending upon the attribute being sensed, in some instances a given sensor modality identified within FIG. 10 may include multiple sensing components while in some instances, a given sensor modality may include a single sensing component. Moreover, in some instances, a given sensor modality identified within FIG. 10 may include power circuitry, monitoring circuitry, and/or communication circuitry and/or other internal electronics 142. However, in some instances a given sensor modality in FIG. 10 may omit some power, monitoring, and/or communication circuitry but may cooperate with such monitoring or communication circuitry located elsewhere.

In some examples, a pressure sensor 144 may sense pressure associated with respiration and may be implemented as an external sensor and/or an implantable sensor. In some instances, such pressures may include an extrapleural pressure, intrapleural pressures, etc. For example, one pressure sensor 144 may comprise an implantable respiratory sensor, such as that disclosed in U.S. Patent Publication No. 2011/0152706, published on Jun. 23, 2011, entitled “METHOD AND APPARATUS FOR SENSING RESPIRATORY PRESSURE IN AN IMPLANTABLE STIMULATION SYSTEM”, the entire teachings of which is incorporated herein by reference in its entirety.

In some examples, a pressure sensor 144 may sense sound and/or pressure waves at a different frequency than occur for respiration (e.g., inspiration, exhalation, etc.). In some instances, this data may be used to track cardiac parameters of patients via a respiratory rate and/or a heart rate. In some instances, such data may be used to approximate electrocardiogram information, such as a QRS complex. In some instances, the detected heart rate is used to identify a relative degree of organized heart rate variability, in which organized heart rate variability may enable detecting apneas or other sleep disordered breathing events, which may enable evaluating efficacy of sleep disordered breathing.

In some examples, pressure sensor 144 comprises piezoelectric element(s) and may be used to detect sleep disordered breathing (SDB) events (e.g., apnea-hypopnea events), to detect onset of inspiration, and/or detection of an inspiratory rate, etc. Although examples are not so limited and may comprise of variety of different types of IMDs.

As shown in FIG. 10, in some examples one sensor modality includes air flow sensor 136, which may be used to sense respiratory information, sleep disordered breathing-related information, sleep quality information, etc. In some instances, air flow sensor 136 detects a rate or volume of upper respiratory air flow.

As shown in FIG. 10, in some examples one sensor modality includes impedance sensor 135. In some examples, impedance sensor 135 may be implemented in some examples via various sensors distributed about the upper body for measuring a bio-impedance signal, whether the sensors are internal and/or external. In some examples, the impedance sensor 135 senses an impedance indicative of an upper airway collapse.

In some instances, the sensors are positioned about a chest region to measure a trans-thoracic bio-impedance to produce at least a respiratory waveform.

In some instances, at least one sensor involved in measuring bio-impedance may form part of a pulse generator, whether implantable or external. In some instances, at least one sensor involved in measuring bio-impedance may form part of a stimulation element and/or stimulation circuitry. In some instances, at least one sensor forms part of a lead extending between a pulse generator and a stimulation element.

In some examples, impedance sensor 135 is implemented via a pair of elements on opposite sides of an upper airway. Some example implementations of such an arrangement are further described herein.

In some examples, impedance sensor 135 may take the form of electrical components not used in an IMD. For instance, some patients may already have a cardiac care device (e.g., pacemaker, defibrillator, etc.) implanted within their bodies, and therefore have some cardiac leads implanted within their body. Accordingly, the cardiac leads may function together or in cooperation with other resistive/electrical elements to provide impedance sensing.

In some examples, whether internal and/or external, impedance sensor(s) 135 may be used to sense an electrocardiogram (ECG) signal.

In some examples, impedance sensor 135 is used to detect SDB events (e.g., apnea-hypopnea events), to detect onset of inspiration, and/or detection of an inspiratory rate, etc.

As shown in FIG. 10, in some examples one sensor modality includes an acceleration sensor 143. In some instances, acceleration sensor 143 is generally incorporated within or associated with the IMD. For instance, in some examples of an IMD, a housing (e.g., can) contains numerous components such as control circuitry, stimulation, and also may contain the acceleration sensor 143 within the housing. However, in some examples, the acceleration sensor 143 may be separate from, and independent of, the IMD. In some examples, acceleration sensor 143 may enable sensing body position, posture, and/or body motion regarding the patient, which may be indicative of behaviors and/or externally induced data from which identification of the presence-absence state of an MRI system may be determined. In some instances, body posture/position is sensed via at least the acceleration sensor 143 and is used to detect the presence-absence state of the MRI system. In some instances, body motion, body posture, and vibration data is sensed by the acceleration sensor 143, as previously described.

Among other uses, the data obtained via the acceleration sensor 143 may be employed to adjust a data model used to identify the presence-absence state of the MRI system and/or therapy provided by the IMD.

In some examples, acceleration sensor 143 enables acoustic detection of cardiac information, such as heart rate via motion of tissue in the head/neck region, similar to ballistocardiogram and/or seismocardiogram techniques. In some examples, measuring the heart rate includes sensing heart rate variability. In some examples, acceleration sensor 143 may sense respiratory information, such as but not limited to, a respiratory rate. In some examples, whether sensed via an acceleration sensor 143 alone or in conjunction with other sensors, one may track cardiac information and respiratory information simultaneously by exploiting the behavior of the cardiac signal in which a cardiac waveform may vary with respiration.

In some examples, acceleration sensor 143 is used to detect SDB events (e.g., apnea-hypopnea events), to detect onset of inspiration, and/or detection of an inspiratory rate, etc. In some examples, the acceleration sensor 143 may be used to detect SDB events during the sleep period and/or may be used continuously to detect arrhythmias. In various examples, the acceleration sensor 143, detection of cardiac information, and/or detection of SDB events may be implemented as described within U.S. Patent Publication No. 2019/0160282, published on May 30, 2019, entitled “ACCELEROMETER-BASED SENSING FOR SLEEP DISORDED BREATHING (SDB) CARE”, and/or U.S. patent application Ser. No. 16/977,664 filed on Sep. 2, 2020, and entitled “RESPIRATION DETECTION”, the entire teachings of which are each incorporated herein by reference in their entirety.

In some examples, an electromagnetic field sensor(s) 131 enables sensing of and/or distinguishing between different types of electromagnetic fields. The electromagnetic fields may include RF fields, static magnetic fields and time-varying gradient magnetic fields, as previous described. The electromagnetic field sensor 131 may comprise one or more implantable sensors, such as an MRI-sensitive conductive element, a Hall effect sensor, a reed switch, a magnetometer, and/or a giant magnetoresistance sensor.

In some examples, radio frequency (RF) sensor 138 shown in FIG. 10 enables non-contact sensing of various additional physiologic parameters and information, such as but not limited to respiratory information, cardiac information, motion/activity, and/or sleep quality. In some examples, RF sensor 138 enables non-contact sensing of additional physiologic data. In some examples, RF sensor 138 determines chest motion based on Doppler principles. The RF sensor 138 may be embodied as the electromagnetic field sensor 131, in some examples.

In some examples, one sensor modality may comprise an optical sensor 132 as shown in FIG. 10. In some instances, optical sensor 132 may be an implantable sensor and/or external sensor. For instance, one implementation of an optical sensor 132 comprises an external optical sensor for sensing heart rate and/or oxygen saturation via pulse oximetry. In some instances, the optical sensor 132 enables measuring oxygen desaturation index (ODI).

As shown in FIG. 10, in some examples one sensor modality comprises an EMG sensor 139, which records and evaluates electrical activity produced by muscles, whether the muscles are activated electrically or neurologically. In some instances, the EMG sensor 139 is used to sense respiratory information, such as but not limited to, respiratory rate, apnea events, hypopnea events, whether the apnea is obstructive or central in origin, etc. For instance, central apneas may show no respiratory EMG effort.

In some instances, the EMG sensor 139 may comprise a surface EMG sensor while, in some instances, the EMG sensor 139 may comprise an intramuscular sensor. In some instances, at least a portion of the EMG sensor 139 is implantable within the patient's body and therefore remains available for performing electromyography on a long term basis.

In some examples, one sensor modality may comprise ECG sensor 140 which produces an ECG signal. In some instances, the ECG sensor 140 comprises a plurality of electrodes distributable about a chest region of the patient and from which the ECG signal is obtainable. In some instances, a dedicated ECG sensor(s) 140 is not employed, but other sensors such as an array of impedance sensors 135 (e.g., bio-impedance sensors) are employed to obtain an ECG signal. In some instances, a dedicated ECG sensor(s) is not employed but ECG information is derived from a respiratory waveform, which may be obtained via any one or several of the sensor modalities in sensor type 130 in FIG. 10.

In some examples, an ECG signal obtained via ECG sensor 140 may be combined with respiratory sensing (via pressure sensor 144 or impedance sensor 135) to determine minute ventilation, as well as a rate and phase of respiration. In some examples, the ECG sensor 140 may be exploited to obtain respiratory information.

In some examples, ECG sensor 140 is used to detect SDB events (e.g., apnea-hypopnea events), to detect onset of inspiration, and/or detection of an inspiratory rate, etc.

As shown in FIG. 10, in some examples one sensor modality includes an ultrasonic sensor 133. In some instances, ultrasonic sensor 133 is locatable in close proximity to an opening (e.g., nose, mouth) of the patient's upper airway and via ultrasonic signal detection and processing, may sense exhaled air to enable determining respiratory information, sleep quality information, sleep disordered breathing information, etc.

In some examples, acoustic sensor 141 comprises piezoelectric element(s), which sense acoustic vibration. In some implementations, such acoustic vibratory sensing may be used to detect sounds caused by fields exerted by the MRI system, SDB events (e.g., apnea-hypopnea events), to detect onset of inspiration, and/or detection of an inspiratory rate, etc.

In some examples, data via sensor types 130 in FIG. 10, such as but not limited to motion and electromagnetic field data, may be used in a training mode of the IMD to correlate various patterns in the sensed information with the identified presence-absence state of an MRI system.

FIGS. 11A-11D are block diagrams schematically illustrating an example MRI engine 27 of an IMD system. As shown by FIG. 11A, the MRI engine 27 may include a plurality of sub-engines 215, 240, 270, 285 which provide inputs to the MRI engine 27 for identifying a presence-absence state of the MRI system.

The MRI engine 27 may include a movement sub-engine 215 used to determine body motion data 220 and posture data 230. As previously described, the body motion data 220 and posture data 230 may be determined from forces sensed from an acceleration sensor. The body motion data 220 and posture data 230 may comprise patient-volitional data, or a combination of patient-volitional data and patient non-volitional data, as previously described at least in connection with FIG. 3C.

FIG. 11B illustrates an example of a movement sub-engine 215. As shown, the movement sub-engine 215 may be used to detect, determine or designate body motion data 220 and posture data 230 based upon the data sensed by the at least one implantable sensor. The body motion data 220 and posture data 230 may be indicative of a pattern of motion or movement of the patient. For example, the body motion data 220 may comprise information related to the type of motion 222, the intensity of the motion 224, and the duration of the motion 226. The posture data 230 may similarly include the type of posture 232 and the duration of the posture 234. The movement engine 210 may identify a pattern, such as an order of motion(s) and posture(s) 228.

The movement sub-engine 215 may determine body motion 220 of the patient, such as determining whether the patient is active or at rest. In some examples, when a vector magnitude of the acceleration measured via the acceleration sensor meets or exceeds a threshold (optionally for a period of time), the measurement may indicate the presence of non-gravitational components indicative of body movement. In some examples, the threshold is about 1.15G. Conversely, measurements of acceleration of about 1G (corresponding to the presence of the gravitational components only) may be indicative of rest. In further examples, an additional threshold or thresholds may be used to distinguish between patient-volitional (e.g., induced) movement, such as walking or running, and patient non-volitional movement, such as MRI-induced movement. The additional threshold(s) may be higher than the threshold for determining the patient is active or at rest.

The movement sub-engine 215 may determine posture 230, including the type of posture 232, by determining whether at least an upper body portion (e.g., torso, head/neck) of the patient is in a generally vertical position (e.g., upright position) or lying down. In some examples, a generally vertical position may comprising standing or sitting. In some examples, this determination may observe the angle of the acceleration sensor between the Y axis and the gravitational vector, which sometimes may be referred to as a y-directional cosine. In some examples, when such an angle is less than 40 degrees, the measurement suggests the patient is in a generally vertical position.

In further examples, the movement sub-engine 215 determines the posture data 230 by rejecting non-posture components from an acceleration sensor via low pass filtering relative to each axis of the multiple axes of the acceleration sensor. In some examples, posture is at least partially determined via detecting a gravity vector from the filtered axes.

In some examples, if the measured angle (e.g., a y-directional cosine) is greater than 40 degrees, then the measured angle indicates that the patient is lying down. In this case, one example posture classification implemented by the movement sub-engine 215 includes classifying sub-postures, such as whether the patient is in a supine position, a prone position, or in a lateral decubitus position. In some non-limiting examples, after confirming a likely position of lying down, the movement sub-engine 215 determines if the patient is in a supine position or a prone position.

Although the above examples describe use of a y-directional cosine to determine a patient position or posture, examples are not so limited. In some examples, a dot product of the vectors may be used, such as with three dimensional vectors. A resulting dot product below a threshold, such as 0.4, may indicate that the patient is lying down.

In some examples, the movement sub-engine 215 is programmed to distinguish between a supine sleep position and a generally supine reclined position. As a point of reference, a generally supine reclined position may be one in which the patient is on a recliner, on an adjustable-type bed, laying on a couch, or the like and not attempting to sleep (e.g., watching television) as compared to sleeping in bed or lying on a tray of an MRI system. An absolute vertical distance between the head and torso of the patient in the supine sleep position is less than the absolute vertical distance between the head and torso in the generally supine reclined position.

Alternatively or in addition, in some examples, the movement sub-engine 215 is programmed to consider or characterize a position of the patient's neck and/or head and/or body position (e.g., as part of a determination of the patient's rotational position while lying down). For example, the movement sub-engine 215 is programmed to estimate a position of the patient's neck based on body position. A determination that the patient's torso is slightly offset may imply different head positions. In some non-limiting examples, the systems and methods of the present disclosure may consider or characterize a position of the patient's neck and/or head via information from a sensor provided with a microstimulator that is implanted in the patient's neck or in conjunction with a sensor integrated into the stimulation lead. In some examples, two (or more) acceleration sensors may be provided, each implanted in a different region of the patient's body (e.g., torso, head, neck) and providing information to the movement sub-engine 215 sufficient to estimate neck and/or head and/or body positions of the patient.

The above explanations provide a few non-limiting examples of some posture determination or designation protocols implemented by the movement sub-engine 215. However, examples are not so limited and a number of other posture determination or designation techniques are also envisioned by the present disclosure, and may be function of the format of the implantable sensor and/or other information provided by one or more additional sensors. For example, various body postures and sub-postures may be determined or designated as implemented and described within U.S. patent application Ser. No. 16/978,275, filed Sep. 4, 2020, and entitled “SYSTEMS AND METHODS FOR OPERATING AN IMPLANTABLE MEDICAL DEVICE BASED UPON SENSED POSTURE INFORMATION”, the entire teachings of which is incorporated herein by reference in its entirety.

As noted above, some systems and methods of the present disclosure may comprise calibrating data sensed to compensate, account, or address the possibility that a position of the at least one implantable sensor (from which posture determinations may be made) within the patient's body is unknown and/or has changed over time (e.g., migration, temporary re-orientation due to change in the implant pocket with changing posture as mentioned above). In some examples, the movement sub-engine 215 is programmed (e.g., with an algorithm) to perform such calibration, such as when the patient is determined to be walking because such a behavior is consistent with a gravity vector (e.g., of an acceleration sensor) pointing downward. In some examples, the movement sub-engine 215 is programmed to perform a calibration, such as via measuring a gravity vector in at least two known patient orientations, of the implantable sensor/accelerometer orientation. In some examples, where the output of the implantable sensor is employed to detect postures of the patient in terms of the body coordinate system of the patient and the orientation of the implantable sensor is such that the implantable sensor axes are not aligned with the body axes, a calibration may be applied to information provided by the implantable sensor to a correct or account for this misalignment.

As an example, the calibration may be based on the movement sub-engine 215 establishing or creating a vertical baseline gravity vector. For example, the vertical baseline gravity vector may be determined by the movement sub-engine 215 during times when the patient is deemed to be likely by upright based on various information, such as information from the implantable sensor, information from other sensors, time of day, patient history, etc., the likelihood or probability that the patient is upright and/or is engaged in an activity in which the patient is likely to be upright (e.g., walking) may be determined, and may be determined as a time average value during periods of higher activity. Once established, the vertical baseline gravity vector may be utilized by the movement sub-engine 215 to calibrate subsequently-received information from the implantable sensor. The vertical baseline gravity vector may be determined/re-set periodically (e.g., at pre-determined intervals).

Although examples are not so limited, and the calibration may be based on establishing a horizontal baseline gravity plane, establishing or creating a vertical baseline gravity vector and a horizontal baseline gravity plane, and/or receiving a predetermined vertical baseline gravity vector and one or more predetermined horizontal baseline gravity vectors, based upon respiratory and/or cardiac waveform polarity information provided by or derived from the implantable sensor, among other variations as described within U.S. patent application Ser. No. 16/978,275, filed Sep. 4, 2020, and entitled “SYSTEMS AND METHODS FOR OPERATING AN IMPLANTABLE MEDICAL DEVICE BASED UPON SENSED POSTURE INFORMATION”, the entire teachings of which is incorporated herein by reference in its entirety.

As shown FIG. 11A, the MRI engine 27 further includes an electromagnetic fields sub-engine 240. The electromagnetic fields sub-engine 240 may identify data indicative of RF fields 267, static magnetic fields 250 and gradient magnetic fields 260. The electromagnetic fields 267, 250, 260 may comprise patient non-volitional data, as previously described at least in connection with FIG. 3C.

FIG. 11C illustrates an example of an electromagnetic fields sub-engine 240. As shown, the electromagnetic fields sub-engine 240 may be used to detect, determine or designate RF field data 267, static magnetic field data 250 and gradient magnetic field data 260 based upon the data sensed by the at least one implantable sensor. The RF field data 267, static magnetic field data 250 and gradient magnetic field data 260 may be indicative of a pattern of electromagnetic fields exerted by an MRI system. For example, the static magnetic field data 250 may include information related to the type of electromagnetic field 254, the intensity of the static magnetic field 252, the duration of the static magnetic field 256, and the sequence or order of the static magnetic field(s) 258. The gradient magnetic field data 260 may include information related to the type of electromagnetic field 264, the intensity of the gradient magnetic field 262, the duration of the gradient magnetic field 266, and the sequence or order of the gradient magnetic field(s) 268. The RF field data 267 may include information related to the type of electromagnetic field 273, the intensity of the RF field 269, the duration of the RF field 271, and the sequence or order of the RF field(s) 272. The electromagnetic fields sub-engine 240 may identify a pattern, such as an order of the electromagnetic fields 265. The electromagnetic fields may be detected, determined or designated using data sensed from an acceleration sensor (e.g., vibrations), a MRI-sensitive conductive element (e.g., electrical signals, such as voltages), and/or another electromagnetic sensor that may sense electromagnetic fields (e.g., magnetometer, Hall effect sensor, etc.).

In some examples, the MRI engine 27 may further include a physiological data sub-engine 270. The physiological data sub-engine 270 may collect additional physiological data, such as cardiac data 275 and/or respiratory data 280, while the MRI system is detected as being present, as further decried herein. The additional physiological data may be used to verify the detected presence-absence state of the MRI system and/or to log events during the identified presence-absence state of the MRI system, such as during an identified presence and/or a non-absence of the MRI system. The additional physiological data may comprise patient-volitional data, as previously described at least in connection with FIG. 3C.

In some examples, the MRI engine 27 may further include other sub-engines, as illustrated by the submodule 285. The sub-engine 285 may include one or more engines which are used to determine different inputs to the MRI engine 27. The other inputs may include a temporal parameter, such as the time of the day 286, time of the year 287, time zone 288, and/or patterns of activity 289.

FIG. 11D illustrates an example of a pattern of electromagnetic fields identified by an MRI engine 27. As shown the pattern 290 includes a sequence of different types of fields 291, 293, 295, 297 exerted by an MRI system for different durations. For example, the sequence includes RF pulses 291, time-varying gradient magnetic fields 293, 295, 297 and gaps between the electromagnetic fields 291, 293, 295, 297. Gaps include periods of time during the MRI scan that there are no gradient magnetic fields and/or RF fields, e.g., pulses 292-1, 292-2, 292-3, 292-3, 292-4, 292-5, 292-6, and 292-M, herein referred to generally as “the pulses 292” for ease of reference. The pattern 290 may identify durations or lengths of time of the gaps and placement of the gaps. For example, the gaps may be between respective time-varying gradient magnetic fields (e.g., pulses 292-2, 292-3, 292-5, 292-M), between RF pulses (e.g., pulses 292-1, 292-4, 292-6) and/or between one of the time-varying magnetic fields and the RF pulses. The timeline 296 in FIG. 11D illustrates the example pattern of the pulses 292 and the gaps 294-1, 294-2, 294-3, 294-P, herein generally referred to as “the gaps 294” for ease of reference, as well as the duration or length of time of the pulses 292, and the duration or length of time of the gaps 294. The pulses 292 correspond to the pulses P₁-P_{M} in the timeline 296. For example, P₁ corresponds to pulse 292-1, P₂ corresponds to pulse 292-2, P₃ corresponds to pulse 292-3, P₄ corresponds to pulse 292-4, etc. The pattern of RF pulses and time-varying gradient magnetic field pulses, including the order of the pulses 292, the length of the pulses 292, and the order and length of the gaps 294, may be recognized by the MRI engine 27 of the IMD or IMD system as a pattern distinctive of an MRI system, alone or in combination with other sensed data.

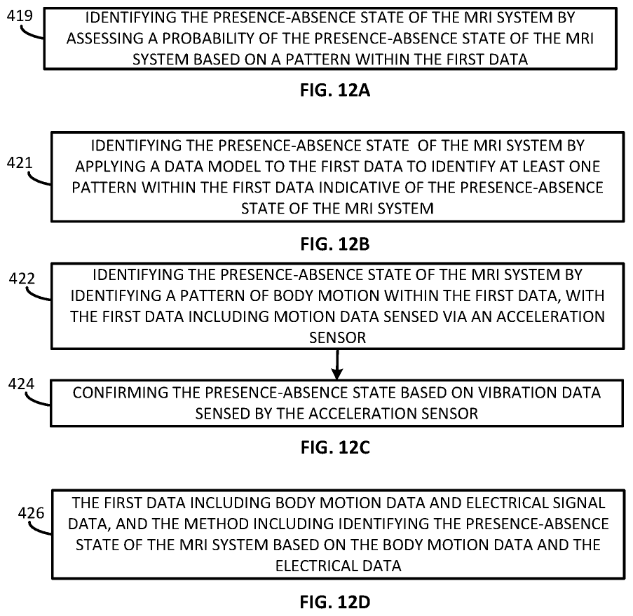

FIGS. 12A-12D are diagrams, which may comprise part of a flow diagram in an example method (e.g., method 10). As shown at 419 in FIG. 12A, identifying the presence-absence state of the MRI system may comprise assessing a probability of the presence-absence state of the MRI system based on a pattern within the first data. In some examples, the probability is determined prior to the MRI system performing an MRI scan of the patient, such as prior to the IMD being exposed to gradient magnetic fields and/or RF pulses exerted by the MRI system. The patterns may include patterns indicative of a likelihood (e.g., a probability) of the presence of the MRI system and patterns indicative of a likelihood (e.g., a probability) of the absence of the MRI system (e.g., patterns likely to include other types of activities and/or patterns indicative of the MRI scan being complete). Example patterns may comprise a pattern of motion, a pattern of posture, a pattern of electromagnetic fields (e.g., vibrations sensed by the acceleration sensor, electrical signals on the MRI-sensitive conductive element, and/or electromagnetic fields sensed via an electromagnetic field sensor, such as a magnetometer, Hall effect sensor, etc. and combinations thereof), a pattern of motion and posture, and a pattern of motion, posture, and electromagnetic fields, and various other combinations thereof.

As may be appreciated, motion patterns may include an identified lack of motion. Example patterns may include a sequence (e.g., order) of motions, a sequence of postures, a sequence of vibrations, a sequence of electrical signals (e.g., voltages induced on internal electronic components and/or the MRI-sensitive conductive element), a sequence of electromagnetic fields, an intensity, type and/or order of one or more of the motions, vibrations, electrical signals, and electromagnetic fields, and a duration or length of time of or between one or more of the motions, postures, vibrations, electrical signals, and electromagnetic fields. As shown at 421 in FIG. 12B, identifying the presence-absence state of the MRI system may comprise applying a data model to the first data to identify the at least one pattern within the first data indicative of the presence-absence state of the MRI system. The data model may be applied to the first data and second data, such as external input data (e.g., time of day, time zone, time of year, and an activity pattern of the patient).

In some examples, the method may comprise, as shown at 422 in FIG. 12C, identifying the presence-absence state of the MRI system by identifying a pattern of body motion within the first data, with the first data including motion data sensed via an acceleration sensor, and as shown at 424 in FIG. 12C, confirming the presence-absence state based on vibration data sensed by the acceleration sensor. The pattern of body motion may be indicative of an initiation of an MRI scan by an MRI system, and used to identify the presence-absence state of the MRI system, such as in method 10 illustrated by FIG. 1. In such examples, the presence-absence state may comprise a presence of the MRI system (e.g., a present state). In related examples, as shown in FIG. 12D, the first data may include body motion data and electrical signal data, such as voltage on an MRI-sensitive conductive element, and as shown at 426, the method may comprise identifying the presence-absence state of the MRI system based on the body motion data and the electrical signal data. The electrical signal data may be caused by the electromagnetic fields from the MRI system during the MRI scan.

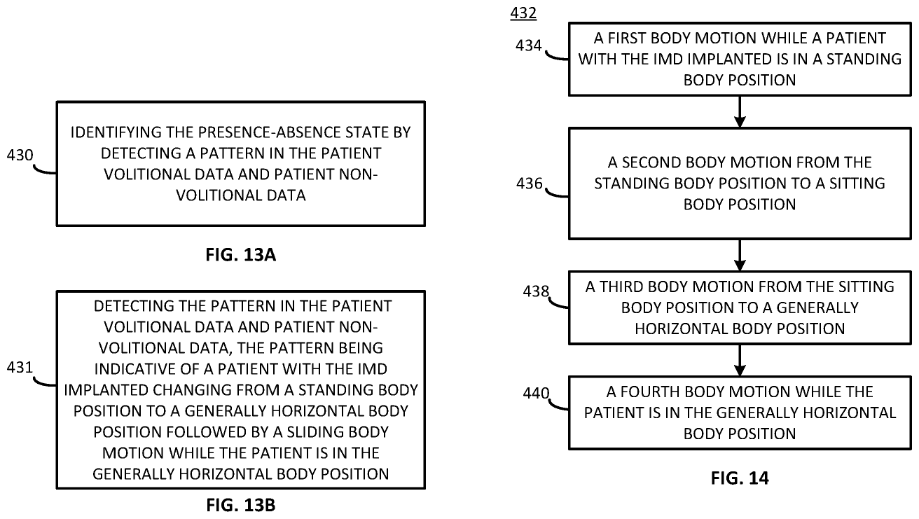

FIGS. 13A-13B are flow diagrams, which may comprise part of a flow diagram in an example method (e.g., method 10). As shown by 430 in FIG. 13A, identifying the presence-absence state of the MRI system may comprise detecting a pattern in the first data including the patient-volitional data and the patient non-volitional data. The pattern may be indicative of a presence and/or a non-absence of the MRI system, in various examples. In some examples, the pattern comprises an order and a type of the patient-volitional data and the patient non-volitional data. As shown by 431 in FIG. 13B, the method may comprise detecting the pattern in the patient-volitional data and the patient non-volitional data, the pattern being indicative of the patient changing from a standing body position to a generally horizontal body position (e.g., patient laying down on the tray of the MRI system), followed by a sliding body motion while the patient is in the generally horizontal body position (e.g., the tray slides into the bore). The generally horizontal body position may comprise one of a supine body position, a prone body position, or a lateral decubitus position which may be followed by the sliding body motion for a first period of time followed by minimal (or no) body motion for a second period of time. For example, for identifying a presence of the MRI system, the pattern may be indicative of a patient siting on the tray of MRI system and then laying down, followed by the tray sliding into the bore of the MRI system while the patient is on the tray. The sliding body motion may be predominantly perpendicular to the gravity vector and/or occurs when a dot product is below a threshold, as described above. The second period of time may be indicative of an amount of time for an MRI scan and is longer than the first period of time, which is associated with the patient being moved into the bore of the MRI scanner.

Such patterns may be used by the IMD system to distinguish from other motion patterns which may occur when the patient is lying down, such as when riding on a train, when on a medical stretcher, or when working on car and using a mechanic's creeper. In some examples, the sliding motion may be at a generally fixed rate of motion. In various examples, although not illustrated, an absence of the MRI system (e.g., the IMD being sufficiently far away from the MRI system) may subsequently be identified by an additional sliding body motion when the patient is lying down (e.g., the tray is sliding out of the bore) and which is followed by or concurrently occurs with a change in the strength of one or more electromagnetic fields and/or a change in body position (e.g., the patient gets off the tray and is standing).

FIG. 14 illustrates an example pattern that comprises a sequence 432 of patient-volitional data and the patient non-volitional data. As shown at 434, the sequence 432 includes a first body motion while a patient with the IMD implanted is in a standing body position (e.g., the patient physically moves into the room with the MRI system). As shown at 436, the sequence 432 includes a second body motion from the standing body position to a sitting body position (e.g., the patient sits down on the tray of the MRI system). As shown at 438, the sequence 432 includes a third body motion from the sitting body position to a generally horizontal body position (e.g., the patient twist or rotates their body on the tray and moves to lay down on the tray). As shown at 440, the sequence 432 includes a fourth body motion while the patient is in the generally horizontal body position, such as sliding body motion (e.g., the tray slides into the bore). However, the examples are not so limited, and the motion pattern may comprise further body motions and/or postures of the patient detected prior to the patient entering the room with the MRI (e.g., the patient checking in and siting down in the waiting room, followed by standing and walking into the room with the MRI system), a general lack of motion after the sliding body motion (e.g., while the MRI scan is occurring), and/or additional body motions and/or postures after the MRI scan.

At least some example methods, systems, and/or devices may involve programming an IMD (e.g., IMD 22 in FIG. 2A) to identify the presence-absence state of an MRI system via at least one implantable sensor, such as an implantable acceleration sensor (e.g., 25A of FIG. 3C, 110 of FIG. 5A, etc.), which may form part of or be associated with the IMD. In some examples, such programming may comprise determining which internally sensed data is correlated with, and/or acts as a surrogate for, information typically used to identify the presence-absence state of the MRI system, such as the above identified patterns of data sensed by the at least one implantable sensor. In at least some examples, the programming may include or involve a data model. In some examples, external circuitry may determine the above identified patterns and program the IMD using the identified patterns, such as by constructing a data model and programming the data model. In other examples, the IMD determines the identified patterns and/or determines the patterns in combination with external circuitry.

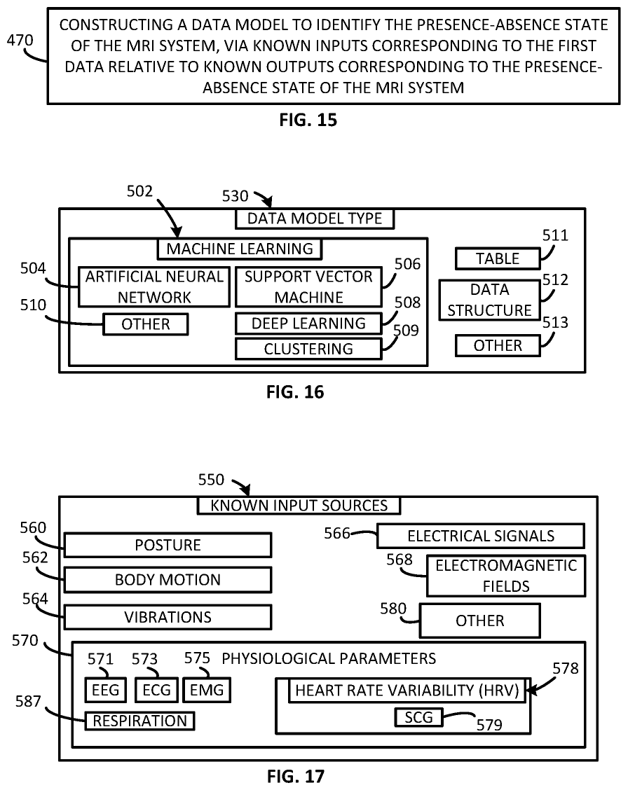

With this in mind, the following example implementations in FIGS. 15-21 provide a framework of parameters, inputs, input sources, outputs, signals, devices, methods, etc., as part of providing an IMD to identify the presence-absence state of an MRI system via internally sensed data. Some of the example implementations comprise a data model or parameters, inputs, etc. associated with use of a data model, while some example implementations omit use of a data model. Regardless of whether a particular example includes a data model or not, it will be understood that the various parameters, inputs, input sources, signals, devices, methods may be combined in various permutations to achieve a desired array of inputs, outputs, etc. by which the IMD may be programmed or otherwise constructed to identify the presence-absence state of an MRI system via internally sensed data.

FIG. 15 is a block diagram, which may comprise part of a flow diagram in an example method (e.g., method 10). As shown at 470, the method may include constructing a data model to identify the presence-absence state of the MRI system via known inputs corresponding to at least the first data relative to known outputs corresponding to at least the presence-absence state of the MRI system. In some such examples, the data model may be constructed via training the data model.

In some examples, the data model may comprise at least one of the data model types 530 shown in FIG. 16. Accordingly, as shown in FIG. 16, in some examples the data model types 530 may comprise a machine learning model 502, which may comprise an artificial neural network 504, support vector machine (SVM) 506, deep learning 508, cluster 509, and/or other models 510. However, examples are not limited to machine learning models 502 and may include a correlation table 511, a data structure 512, among other models 513, and which may include the above described patterns and/or a probabilistic approach, which may be known inputs.

In some examples, the artificial neural network 504 may estimate a function(s) that depend on inputs. In some such examples, one or more layers of artificial neurons may receive input data and generate output data. The inputs and outputs may comprise the data sensed by the at least one implantable sensor and/or functions related to such data or other functions. Neural networks may comprise networks such as, but not limited to, learning networks (e.g., deep, deep structured, hierarchical, and the like), convolutional, auto-type networks (e.g., auto-encoder, auto-associator), Diablo networks, and neural network models (e.g., feedforward, recurrent).

In some examples, the SVM 506 may utilize a linear classification. This classification may act to separate the data points into classes based on distance of the data points from a hyperplane. In some examples, the hyperplane is arranged to maximize the distances from the hyperplane to the nearest data points on either side of the hyperplane. This arrangement may group points located on opposite sides of the hyperplane into different classes. However, in some examples, the SVM 506 may comprise a nonlinear classification that separates the data points with a hyperplane in a transformed feature space. The transformed feature space may be determined by one or more kernel functions, including nonlinear kernel functions. In some examples, the SVM 506 is a multiclass SVM that separates data points into more than two classes, which may reduce a multiclass problem into multiple binary classification problems.

In some examples, the deep learning 508 may comprise models such as, but not limited to, convolutional networks (e.g., deep belief, neural), belief networks, Boltzmann machines, deep coding networks, stacked auto-encoders, stacking networks (e.g., deep or tensor deep), hierarchical-deep models, deep kernel machines, and the like. It will be understood that such examples may comprise variants and/or combinations of the above-noted example networks.

In some examples, per type 509, the data model may comprise a clustering method(s), which may comprise hierarchical clustering, k-means clustering, density-based clustering, and the like. In some examples, the hierarchical clustering may be used to construct a hierarchy of clusters of sensed data. In some such examples, the hierarchical clustering utilizes a “bottom up” approach (e.g., agglomerative) wherein each data point starts in its own cluster, and pairs of clusters are merged at progressively higher levels of the hierarchy. However, in some examples, the hierarchical clustering utilizes a top-down approach in which all data points start in one cluster, and then clusters are split at progressively lower levels of the hierarchy.

In some examples, the k-means clustering implementation may comprise placing the sensed data into k clusters, where k is an integer equal or greater than two. Via such clustering, each data point belongs to a cluster having a mean that is closer to the data point than any means of the other clusters. However, in some examples, a machine learning model (MLM) may comprise density-based clustering, which may be used to group together data points that are close to one another, while identifying as outliers any data points that are far away from other data points.

In some examples, as represented per “other” type 510 in FIG. 16, a MLM may comprise a mean-shift analysis that may be used to determine the maxima of a density function based on discrete data sampled from that function.

In some examples, as represented per “other” type 510 in FIG. 16, a MLM may comprise structured prediction techniques and/or structured learning techniques. Such techniques may be used to predict structured objects and/or structured data, such as structured patient-volitional data and patient non-volitional data. In some such examples, such structured prediction and/or structured learning techniques may comprise graphical models, probabilistic graphical models, sequence labeling, conditional random fields, parsing, collective classification, bipartite matching, Bayesian networks or models, and the like. It will be understood that such examples comprise variants and/or combinations of the above-noted example techniques.

In some examples, a MLM may comprise anomaly detection and/or outlier detection that may be used to identify data, such as patient-volitional data and/or patient non-volitional data, that does not conform to an expected pattern or are otherwise distinct from other data in a dataset.

In some examples, machine learning model may comprise learning methods that incorporate a plurality of the machine learning methods.

It will be understood that at least some example methods (and/or devices) of the present disclosure may sense patient-volitional data and patient non-volitional data, and identify the presence-absence state of the MRI system without use of a constructed data model and/or trained data model, such as but not limited to, a machine learning model. Further, the data model may be constructed on a per-patient basis and/or a representative patient basis.

In some examples, a method may comprise implementing (and/or a system or device may implement) construction of a data model at least partially via at least one external resource, in communication with the IMD, according to at least some external data. In some such examples, the external data comprises data or signals of electromagnetic fields (e.g., static, gradient), motion, posture, vibrations, electrical signals, time of day, activity patterns, time zone, time of year, and physiological parameters. In further examples, the external data may comprise one or more known or expected patterns of electromagnetic fields, motion, posture, vibrations, electrical signals, time of day, activity patterns, time zone, time of year, and (additional) physiological parameters and corresponding outputs, such as externally measured data indicative of the presence-absence state of an MRI system.

FIG. 17 is a block diagram schematically representing at least some example known input sources 550. The input sources 550 may comprise external sources and/or internal sources, such as data sensed by the at least one implantable sensor of a particular IMD or a plurality of IMDs. In some example methods, a data model may be constructed via providing known inputs to the data model based on known input sources 550. The known input sources 550 may comprise signals indicative of posture 560, motion 562, vibrations 564, electromagnetic fields 568 including RF fields, static magnetic fields and gradient magnetic fields, electrical signals 566, additional physiological parameters 570, and other inputs 580 including time zone, time of year, and activity patterns. In various examples, the known input sources 550 may comprise data indicative of expected or known patterns of sensed data, such as patterns of motion and posture, as well as electromagnetic fields which are indicative of the presence-absence state (e.g., the presence or absence) of the MRI system, as described above.

The additional physiological parameters 570 may comprise a respiration signal 587, a respiration rate variability signal, a heart rate variability signal 578, in which may be obtained from seismocardiography sensing (SCG) 579, an electroencephalogram (EEG) parameter 571, ECG parameter 573, and/or an EMG parameter 575. Other inputs sources 550 may comprise ballistocardiography sensing (BCG), and/or accelerocardiograph sensing (ACG). In some examples, the SCG, BCG, ACG sensing may be provided via an implanted acceleration sensor or via other types of implantable sensors. In some examples, the additional physiological parameters 570 may be indicative of the presence-absence state of an MRI system, which may be patient specific. For example, a particular patient may experience claustrophobia and may have an increased heartrate when entering the bore of the MRI system. Other patients may exhibit a decrease in heartrate due to lack of activity during the MRI scan.

In various examples, the motion 562 may be used to obtain at least one of the additional physiological parameters 570. For example, motion data sensed by an acceleration sensor may be used to determine respiratory information, as further described herein. In some examples, the respiration information is determined by sensing, via the acceleration sensor, rotational movement associated with a respiratory body portion of the patient with the IMD implanted, with the rotational movement being caused by breathing. In such examples, the respiratory body portion may comprise a chest wall and/or abdominal wall of the patient, and the motion may include chest motion, such as chest wall motion comprising a rotational movement of the chest wall and/or rotational movement of an abdominal wall or portion of the abdomen indicative to respiratory information, and as described within U.S. patent application Ser. No. 16/977,664, filed on Sep. 2, 2020, and entitled “RESPIRATION DETECTION”, the entire teachings of which is incorporated herein by reference in its entirety.

The known input sources 550 may include various external and internal data sources, such as the implantable sensor of the IMD, implantable sensors of other IMDs, external databases which store data from a plurality of IMDs, such as various patient-volitional and patient-non volitional data for the respective IMD or for a plurality of IMDs. Accordingly, the data model may be constructed for the particular patient (e.g., per-patient basis) or representative number of patients (e.g., representative patient basis). Additionally, the data model may be updated overtime using feedback data from the particular IMD and/or a plurality of IMDs.

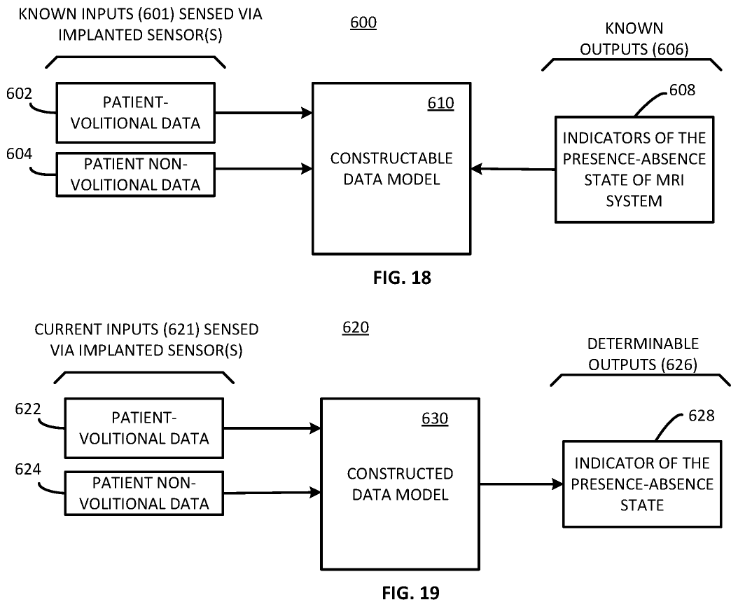

FIG. 18 is a diagram schematically representing an example method 600 of constructing a data model for use in later identifying the presence-absence state of an MRI system. As shown in FIG. 18, the method 600 comprises constructing a data model by providing known inputs 601 and known outputs 606 to the constructable data model 610. The known inputs 601 may be obtained and/or sensed via at least one implanted sensor of a particular IMD and/or via implanted sensors of a plurality of representative IMDs. In some examples, the known outputs 606 may be obtained and/or sensed via at least one sensor located external to the patient's body, herein sometimes referred to as “an external sensor”.

The known inputs 601 may comprise patient-volitional data 602 and patient non-volitional data 604. Example patient-volitional data 602 may comprise motion and posture data sensed using an acceleration sensor and patient non-volitional data 604 may comprise data indicative of electromagnetic fields, such as vibrations caused by the electromagnetic fields and as sensed by the acceleration sensor.

The known outputs 606 may comprise indicators of the presence-absence state of an MRI system 608. For example, the known outputs 606 (e.g., the indicators 608) may comprise data measured externally from the IMD, such as by the at least one external sensor. The at least one external sensor may comprise an acceleration sensor and/or a non-acceleration sensor configured to sense electromagnetic fields and/or other phenomenon (e.g., body motion or vibrations) experienced by the patient when in the presence of the MRI system and in the absence of the MRI system. The acceleration sensor and/or non-acceleration sensor may be an implementation of and/or substantially include the same features and operations of sensors previously described in connection with FIGS. 3C, 5A-9B, and/or 10, but with the acceleration sensor and/or a non-acceleration sensor being external to the patient. The at least one external sensor may be placed on the patient or on another location that is sufficiently close to a location where the patient would experience the electromagnetic fields and/or other phenomenon from the MRI system.

In some examples, the known outputs 606 may comprise indicators from a plurality of data signals obtained by the at least one external sensor prior to an MRI scan and during one or more MRI scans, and with the external sensor at different distances from the MRI system. The electromagnetic fields may radiate out a particular distance, and the known outputs 606 may be used to identify known inputs 601 that are indicative of the IMD being outside a threshold distance (e.g., a safe distance) from the MRI system and within the threshold distance (e.g., at an unsafe distance) from the MRI system in which the electromagnetic fields may impact the IMD, as previously described in connection with FIG. 1.

As previously described, constructing the data model may comprise training a data model, such as one of the data models in data model types 530 in FIG. 16 with one of the example data model types comprising a machine learning model 502. By providing such known inputs 601 and known outputs 606 to the constructable data model 610, a constructed data model 630 (FIG. 19) may be obtained. As noted elsewhere, the constructable data model 610 (FIG. 18) may comprise a trainable MLM and the constructed data model 630 (FIG. 19) may comprise a trained MLM. In the particular example, the constructable data model 610 (FIG. 18) is trained (forming the constructed data model 630) using data from the particular IMD, and may be said to be “per-patient”. However examples are not so limited, and may include constructing a data model that is “representative patient-based”. Once constructed, the data model 603 as illustrated by FIG. 19, may be used in a method 620 in which currently sensed inputs 621 are fed into the constructed data model 630, which produces an output 624 as an indicator 628 of a presence-absence state of the MRI system. The indicator 628 is used to identify the presence-absence state of the MRI system.

FIG. 19 is a diagram schematically representing an example method 620 of using a constructed data model 630 for identifying a presence-absence state of an MRI system using internal measurements, such as via an implanted sensor. As shown in FIG. 19, currently sensed inputs 621 are fed into the constructed data model 630 (e.g., trained MLM), which then produces determinable outputs 626, such as the indicator 628 of the presence-absence state of an MRI system, which is based on the current inputs 621. In some examples, the current inputs 621 include patient-volitional data 622 and patient non-volitional data 624 obtained via the implantable sensor and the current inputs 621 correspond to the types of known inputs 601 obtained via the implantable sensor.

In some examples, just one or some of the 621 may be used, while all of the inputs 621 may be used in some examples.

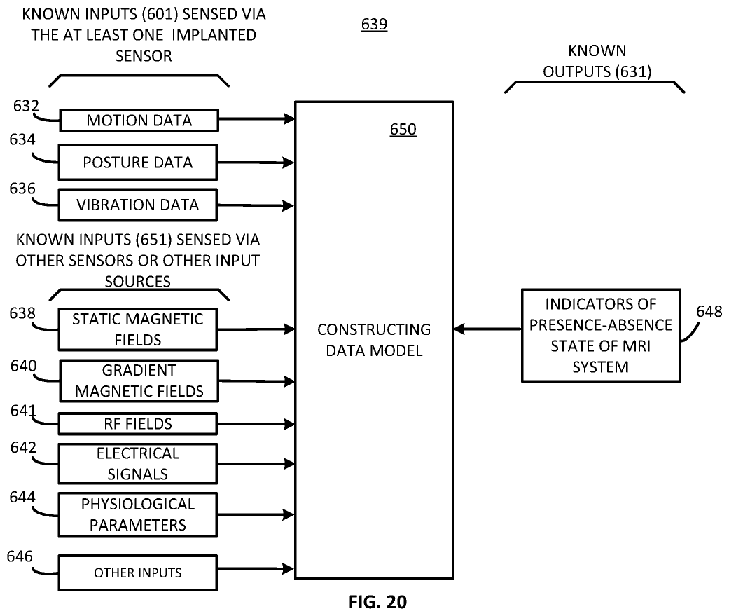

FIG. 20 is diagram schematically representing an example method 639 of constructing a data model. Method 639 may comprise at least some of substantially the same features and attributes as method 600 in FIG. 18, except further comprising additional known inputs 651, e.g., other inputs sensed or otherwise provided by other sensors or input sources. The known outputs 631 may include those previously described in connection with FIG. 18, e.g., the indicators 648 of the presence-absence state of the MRI system. In some examples, the known inputs 601, 651 may be sensed using internal sensors to the IMD. In some examples, the known inputs 601, 651 may further or alternatively include data sensed by external data sources, such as sensors of other IMDs and/or patterns of known inputs that indicate the presence-absence state of the MRI system. In some examples, using both the internally measured known inputs and the externally measured known inputs may enhance accuracy, robustness, etc., in constructing the data model (650).

As shown by FIG. 20, the known inputs 601 sensed via the at least one implantable sensor (e.g., an acceleration sensor) comprise motion data 632, posture data 634, and vibration data 636. In some examples, the motion data 632 and posture data 634 may comprise the patient-volitional data 602 in FIG. 18, and the vibration data 636 may comprise the patient non-volitional data 604 in FIG. 18. The vibration data 636 may be used to determine RF fields, time-varying gradient magnetic fields and/or static magnetic fields. The known inputs 651 sensed via the other sensor circuitry may comprise static magnetic fields 638, time-varying gradient magnetic fields 640, RF fields 641, electrical signals 642, and (additional) physiological parameters 644. The electrical signals 642 may be induced on an internal component of the IMD by electromagnetic fields, as previously described. Additionally, other inputs 646 may be provided to construct the data model, such as a temporal parameter.

In some examples relating to at least FIG. 20, just one or some of the inputs 601 and just some of the inputs 651 may be used, while all of the inputs 601 and/or all of the inputs 651 may be used in various examples.

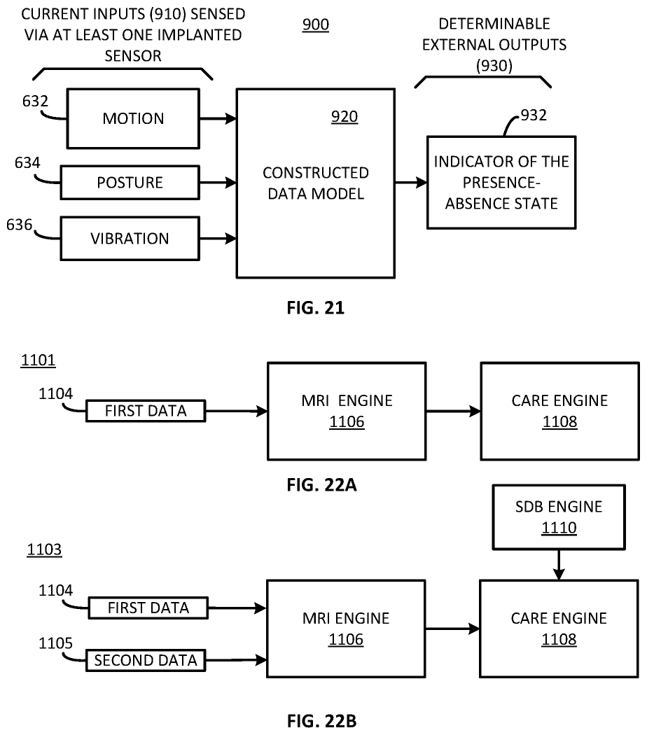

FIG. 21 is a diagram schematically representing an example method 900 of using a constructed data model 920 for identifying the presence-absence state of an MRI system. The constructed data model 920 is obtained via the method 639 in FIG. 20 via constructing data model 650, which includes the additional known inputs 651. As shown in FIG. 21, currently sensed inputs 910 are fed into the constructed data model 920 (e.g., a trained MLM), which then produces a determinable output 930, such as an indicator 932 of the presence-absence state of an MRI system, which is based on the current inputs 910. In some examples, the current inputs 910 are obtained via the at least one implanted sensor (e.g., acceleration sensor), which include motion data 632, posture data 634, and vibration data 636 indicative of electromagnetic fields obtained from the acceleration sensors (or other input sources) and the current inputs 910 correspond to the types of known inputs 601 obtained via the at least one implantable sensor. Although not illustrated, in some examples, the current inputs 910 may additionally comprise at least one input sensed via other sensors or sources, such as those similar to the known inputs 651 in FIG. 20.

FIGS. 22A-22B are block diagrams schematically presenting example IMD systems 1101, 1103 including an MRI engine 1106. The MRI engine 1106 illustrated by FIGS. 22A-22B may comprise the MRI engine 27 that forms an IMD system 20 with an IMD 22 and at least one implantable sensor 25, as illustrated by FIG. 2A. Accordingly, the MRI engine 1106 may identify or determine a presence-absence state of an MRI system using first data 1104 and optionally second data 1105, as previously described.

As noted above, the MRI engine 1106 may be programmed to control one or more operational features of the IMD system based upon an identified presence-absence state of the MRI system (or communicates with another engine or engine programmed to control an operational feature). For example, as shown by FIG. 22A, the IMD system 1101 includes the MRI engine 1106 that communicates with another engine or engine programmed to control an operational feature, such as the illustrated care engine 1108. The control of the feature may comprise enabling and/or disabling a feature of the IMD system 1101 in response to the presence-absence state of the MRI system, such as enabling or disabling performance of therapy, adjusting the care settings, maintaining and/or switching operational modes, etc. The care engine 1108 may provide care to the patient. Providing care may include, but is not limited to, measuring and/or or monitoring physiological data, providing information (e.g., feedback, suggestions, alerts) to the patient or a caregiver based on the physiological data, and/or delivering therapy to the patient, and various combinations thereof.

In an example, the MRI engine 1106 communicates with the care engine 1108 to select or switch an operational mode of the IMD system 1101 (such as an IMD of the IMD system 1101) based upon the identified presence-absence state of the MRI system. The “operational mode” of the IMD or IMD system may include one or more of care parameters, such as stimulation parameters, sensing parameters, timing parameters, diagnostic parameters, and other electrical configurations and/or device settings. As some examples, operational modes may comprise corresponding stimulation therapy settings or mode, such as a stimulation or therapy mode of the IMD or IMD system, a normal-operation mode of the IMD or IMD system, and an MRI mode of the IMD or IMD system and/or adjustments in patient control in addition to or instead of stimulation therapy settings. A therapy mode may comprise delivering therapy to a patient in response to particular parameter or event (e.g., cardiac signals, sleep, respiration values). The selection of the operational mode may thereby include effecting changes to the particular care, such as adjusting a threshold (diagnostic) parameter for initiating (or suspending) delivery of therapy from the IMD, adjusting a sensing parameter, such as timing(s) used for sensing the physiological (or other) data, adjusting a formula used for calculating the physiological data, and/or adjusting a state of internal electronics. For example, in response to identifying or determining the presence-absence state of the MRI system comprises a presence of the MRI system, the MRI engine 1106 may communicate with the care engine 1108 to disable or suspend a normal-operation mode or a stimulation mode of the IMD. In a number of examples, the MRI engine 1106 may change a state of internal electronics to mitigate the effects from the MRI system. For example, in response to identifying or determining the presence of the MRI system and as a safety feature, the IMD may change electrical configuration to reduce induced voltages or temperature increases on internal components of the IMD, such as by shorting electrodes together. In various examples, the MRI engine 1106 communicates with the care engine 1108 to maintain and/or switch operational modes in response to an identified absence of the MRI system (e.g., an absent state), such as a normal or default operation mode.

As an example, the IMD may comprise an SDB device having an IPG. In such examples, stimulation or therapy mode may comprise delivering stimulation therapy (e.g., delivering a stimulation signal) when the patient with the IMD implanted is in a state of sleep. As further illustrated by FIG. 22B, the IMD system 1103 may further comprise an SDB engine 1110 which includes a sleep detection feature to identify SDB. The MRI engine 1106 may disable the sleep detection feature in response to the identified or determined presence-absence state comprising a presence of the MRI system. More specifically, during an MRI scan, the patient may be in a body position and may exhibit a lack of movement such that the SDB engine 1110 may determine the patient is in a state of sleep, and the IMD may enter a stimulation mode. The MRI engine 1106 may disable this feature by identifying or overruling the sleep detection by the SDB engine 1110, and indicating the presence of the MRI system. Disabling performance of the therapy may comprise preventing application of a stimulation signal in response to the identified presence of the MRI system. Non-limiting examples of some features implemented by the SDB engine 1110 in accordance with systems and methods of the present disclosure may comprise at least some of substantially the same features and attributes as described within at least: U.S. Patent Publication No. 2019/0160282, published on May 30, 2019, and entitled “ACCELEROMETER-BASED SENSING FOR SLEEP DISORDED BREATHING (SDB) CARE”; U.S. patent application Ser. No. 16/978,470, filed Sep. 4, 2020, and entitled “SLEEP DETECTION FOR SLEEP DISORDERED BREATHING (SDB) CARE”; and/or U.S. patent application Ser. No. 16/978,283, filed Sep. 4, 2020, and entitled “SYSTEMS AND METHODS FOR OPERATING AN IMPLANTABLE MEDICAL DEVICE BASED UPON SENSED PHYSICAL ACTION”, the entire teachings of which are incorporated herein by reference in their entireties.

In further examples, the care engine 1108 may be enabled in response to the identified presence-absence state of the MRI system, although the care or sleep detection feature of the SDB engine 1110 is disabled. For example, in response to the identified presence of the MRI system by the MRI engine 1106, the care engine 1108 may perform (or continue performing) logging of various events and/or communication of data, as further described herein. Although examples are not so limited and the MRI engine 1106 may perform the logging of events and/or communication of data. Further, as noted above, in some examples, the sensing parameters of the care engine 1108 are adjusted in response to the identified presence of the MRI system.

Examples are not limited to SDB devices and may comprise other neurostimulators, sensing, and/or cardiac care devices. In general terms, with a neurostimulator, neurostimulation may be disabled in response to the identified presence-absence state of the MRI system comprising a presence of the MRI system or the detection criteria for triggering therapy may be adjusted. Other example sensing and/or stimulating devices may be directed to sensing and/or simulating for urinary and/or pelvic disorders.

For cardiac care devices, the device may be switched to an MRI mode in response to the identified presence-absence state of the MRI system comprising a presence of the MRI system (e.g., a present state). For example, in response to identifying the presence of the MRI system, the MRI engine 1106 may communicate with the care engine 1108 to enable an MRI mode of the IMD or IMD system 1101, 1103 in which therapy or stimulation is not suspended. As may be appreciated, with a cardiac care device, it may be desirable to continue to deliver therapy during the MRI scan for the health of the patient. The MRI mode may include adjustments in therapy parameters (e.g., stimulation parameters, sensing parameters, timing parameters), such as the detection criteria for triggering therapy (e.g., diagnostic parameters), and/or changing the state of internal electronics to mitigate the effects from the MRI system, as described above. In addition to or alternatively to the adjustment in therapy parameters, the MRI mode may include adjustments in patient control, such as disabling patient control. As an example, during an MRI scan, the measured cardiac signals may be over or under-sensed due to the presence of the electromagnetic fields. The MRI mode may include changes in the algorithm(s) for monitoring the cardiac signals, such as a change in how arrhythmia is detected. After the MRI scan is complete or the MRI system is absent, the IMD may switch back to the normal-operation mode, in which cardiac signals are monitored as was performed prior to the MRI being presence. Example MRI modes for a cardiac care device may include a fixed-rate or non-demand/asynchronous pacing mode, as opposed to a rate-responsive and/or demand pacing mode during a normal-operation mode.

As may be appreciated, by normal-operation mode, it is meant that the IMD of example IMD systems 1101, 1103 performs functions in a manner that does not specifically take into account the presence of strong electromagnetic fields exerted by an MRI system. For a pacer or other cardiac care device, normal functions may involve any of a variety of cardiac rhythm management functions, such as anti-bradycardia pacing, anti-tachycardia pacing (ATP), overdrive pacing, and the like, that involve delivering electrical stimulation to heart tissue using otherwise conventional techniques. For some IMDs, such as neural stimulators or SDB device, normal functions may involve the delivery of electrical stimulation to nerves or other tissues, in a manner that that does not specifically take into account the presence of the strong electromagnetic fields.

In various examples, the IMD of example IMD system 1101, 1103 may be designed for manually entering the IMD into a MRI mode, such as disabling delivery of stimulation therapy or otherwise adjusting the care provided, the therapy delivered, and/or the detection criteria. More specifically, a caregiver, doctor, or MRI technician may manually enter the IMD into MRI mode prior to the MRI scan. In such examples, the identified presence-absence state of the MRI system may be used as a safety feature, in case the manual adjustment does not occur. Further and/or alternatively, the IMD may collect various data, which may be used to construct or train a data model and/or to revise a constructed data model for entering the IMD into the MRI mode. As a particular example, the IMD may comprise a data model that is on a patient-representative basis and which is updated over time to be on a patient-basis using data sensed by the particular IMD and/or IMD system.

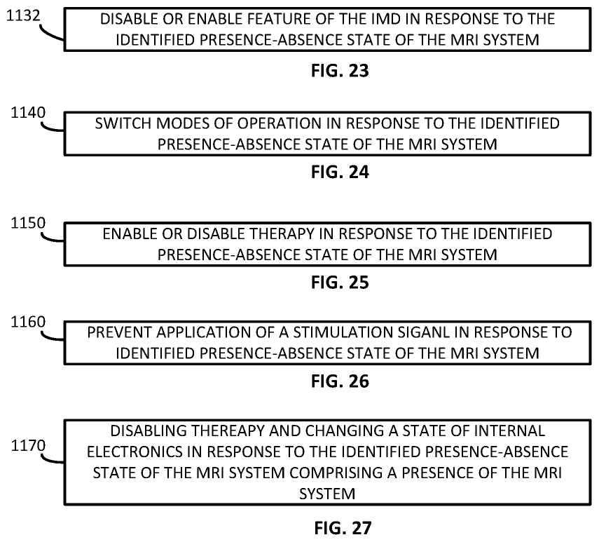

FIGS. 23-35 are diagrams, which may comprise part of a flow diagram in an example method (e.g., method 10). As previously described, one or more features of the IMD may be controlled in response to the identified presence-absence state of the MRI system.

As shown at 1132 in FIG. 23, the method may comprise disabling or enabling a feature of the IMD in response to the identified presence-absence state of the MRI system. More specifically, as shown at 1140 in FIG. 24, the method may comprise switching a mode of operation in response to the identified presence-absence state of the IMD. As an example, at shown at 1150 in FIG. 25, therapy may be enabled or disabled in response to the identified presence-absence state of the MRI system, such as by switching the IMD to a MRI mode of operation in response to an identified presence of the MRI system (e.g., a present state). Disabling the therapy, as shown at 1160 in FIG. 26, may comprise preventing application of a stimulation signal in response to the identified presence-absence state of the MRI system, although examples are not limited. As another example, as shown at 1170 of FIG. 27, the method may comprise disabling the therapy and changing a state of internal electronics or circuit components in response to the identified presence-absence state of the MRI system comprising a presence of the MRI system.

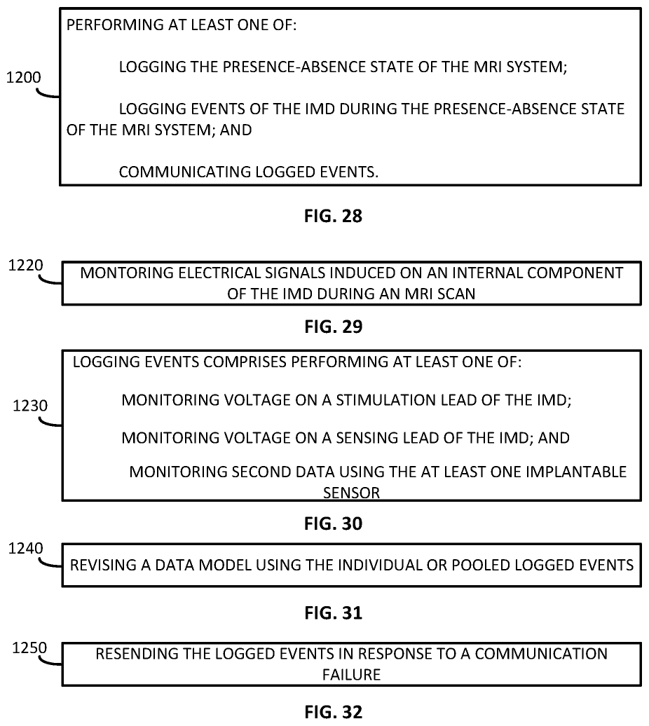

In some examples, the controlled feature(s) may comprise continued performance of therapy and/or logging of data. For example, as shown in 1200 in FIG. 28, the method may comprise performing at least one of logging the presence-absence state of the MRI system (e.g., log that the IMD is exposed to electromagnetic fields from an MRI system), logging events of the IMD during the presence-absence state of the MRI system, and communicating the logged events or presence-absence state of the MRI system to external circuitry. As shown at 1220 in FIG. 29, logging the events may comprise monitoring electrical signals (e.g., voltages, current, and/or impedance) induced on an internal component of the IMD, such as voltages, or impedance on a stimulation lead of the IMD during an MRI scan. In further examples, as shown at 1230 of FIG. 30, logging the events may comprise performing one or more of monitoring voltage(s) on a stimulation lead of the IMD, monitoring voltage(s) on a sensing lead of the IMD, and monitoring second data (e.g., additional data) using the at least one implantable sensor. In some examples, the logging of events is in response to (and during) the identified presence of the MRI system (e.g., during a present state). In response to a voltage on the stimulation lead(s) and/or the sensing lead(s) exceeding a threshold voltage, the IMD may disable one or more of the sensing lead(s) and the stimulation lead(s). For example, in an IMD with multiple stimulation leads, the IMD may disable all but one of the multiple stimulation leads.

In some examples, the second data may comprise data sensed via an acceleration sensor and non-acceleration sensor circuitry. In such examples, at least some of the first data and second data sensed via the acceleration sensor may comprise patient-volitional data and, in some examples, at least some of the first data and second data sensed via the acceleration sensor may comprise patient non-volitional data. The first and second data sensed via the non-acceleration sensor circuitry may comprise patient non-volitional data. The second data and/or logged events may comprise bioimpedance sensed via non-acceleration sensor circuitry, a heartrate, EMG and/or ECG (or other heart signal) sensed via the non-acceleration sensor circuitry, an IPG signal sensed via the non-acceleration sensor circuitry, vibrations sensed via the acceleration sensor (e.g., indicative of electromagnetic fields or of physiological data), RF fields, and static and gradient magnetic fields sensed via the non-acceleration sensor circuitry. As an example, the logged events comprise at least one of respiratory information including a respiratory rate, cardiac information including a heart rate, and body motion. In such examples, logging the events may comprise monitoring chest motion due to respiration of the patient with the IMD implanted.

In various examples, as shown at 1240 of FIG. 31, the method may comprise revising a data model using the individual logged events and/or pooled logged events from a plurality of IMDs, such as the logged events of the methods illustrated by FIGS. 28-30. For example, the constructed data model 920 of FIG. 21 may be revised using the individual logged events and/or pooled logged events from a plurality of IMDs, and/or which may be performed by the MRI engine 1106 of FIG. 22A or 22B and/or by external circuitry, such as the mobile device 1670 and/or patient management tool 160 further illustrated herein by FIG. 41. The logged events and/or data may thereby be used as feedback data to improve a data model used to identify the presence-absence state of the MRI system (e.g., determine whether an MRI system is present or absent).

In related and non-limiting examples, the logged events may be communicated to external circuitry, such as external device 26 illustrated by FIG. 2A. For example, and with reference back to FIG. 2A, the MRI engine 27 and/or other engine of the IMD is programmed to provide information to the patient and/or caregiver relating to the identified presence-absence state of the MRI system 21 or other information of possible interest implicated by information from the at least one implantable sensor 25, such as the logged events. As a point of reference, the IMD 22 may be configured to interface (e.g., via telemetry) with a variety of external devices. For example, the external device 26 may include, but is not limited to, a patient remote, a physician remote, a clinician portal, a handheld device, a mobile phone, a smart phone, a desktop computer, a laptop computer, a tablet personal computer, etc. The logged events and other data captured by the IMD 22 may be used as part of a software application, uploaded to a database or other external storage source (e.g., the cloud, a website), etc. The external device 26 may include a smartphone or other type of handheld (or wearable) device that is retained and operated by the patient to whom the IMD 22 is implanted. In some examples, the external device 26 may include a personal computer or the like that is operated by a medical caregiver for the patient. The external device 26 may include a computing device designed to remain at the home of the patient or at the office of the caregiver.

With the above in mind, the MRI engine 27 may be programmed (or communicates with another module or engine of the IMD system 20 that is programmed) to communicate an alert in response to the logged events. The alert may include an audible notification, such as an alert or an alarm, or vibration provided to the patient and/or a data message communicated to the external circuitry, such as the external device 26. The alert may be provided in response to a detected problem, such as the logged data being indicative of a therapy event and/or a failure of the IMD.

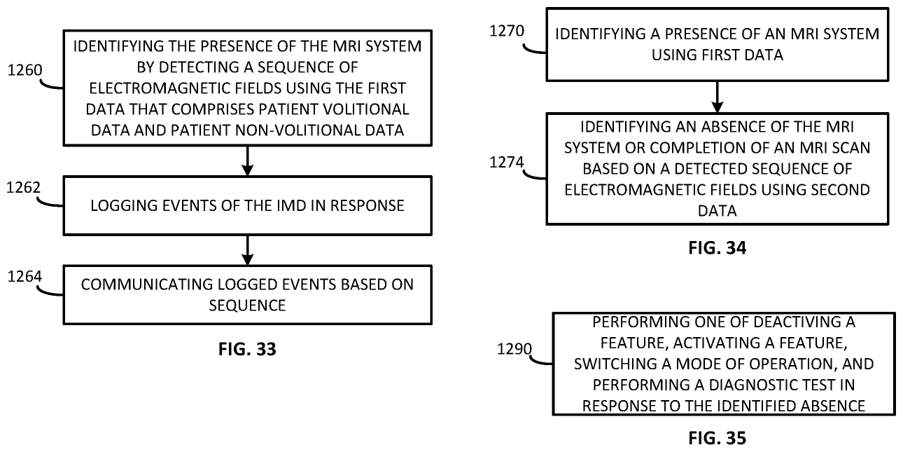

In some examples, the communication may fail due to RF fields from the MRI system when the MRI system is present. In such examples, as shown at 1250 of FIG. 32, the method may comprise resending the logged events in response to the communication failure. The recommunication may be based on a detected pattern or sequence of electromagnetic fields, such as the pattern 290 illustrated by FIG. 11D. The detected pattern or sequence of electromagnetic fields may uniquely identify the presence of the MRI system. For example, as shown by FIG. 33, the method may comprise, as shown at 1260, identifying the presence of the MRI system by detecting a sequence of electromagnetic fields using at least the first data, as shown at 1262, logging the events in response to the identified presence, and as shown at 1264, communicating the logged events based on the detected sequence. The communication may include the first attempt or a recommunication after a failure. Although examples are not so limited, and the communication may be periodically communicated until the communication is successful and without being based on the detected pattern of electromagnetic fields. In some examples, detecting the sequence of electromagnetic fields comprises identification of a type and an order of electromagnetic fields, gaps between electromagnetic fields, and a duration of the electromagnetic fields and duration of the gaps between the electromagnetic fields (e.g., between RF pulses and gradient magnetic fields). The communication of the logged events may be during an anticipated next gap between electromagnetic fields. However, examples are not so limited and may include communicating the logged data in response to identification of an absence of the MRI system and/or completion of an MRI scan.

As described above, the method may further comprise identifying an absence of the MRI system and/or completion of an MRI scan, which may be in addition or alternative to identify the presence of the MRI system. For example, as shown at 1270 in FIG. 34, the method includes identifying a presence of the MRI system using the first data sensed by the at least one implantable sensor and, as shown at 1274, identifying an absence of the MRI system or completion of the MRI scan based on a detected sequence of electromagnetic fields using second data sensed by the at least one implantable sensor. As previously described by FIG. 13B, the presence of the MRI system may be identified by a pattern of movement and/or electromagnetic fields. As an example, the pattern may be indicative of a patient siting on the tray of MRI system and then laying down, followed by first sliding body motion caused by the tray sliding into the bore of the MRI system. The absence of the MRI system or completion of the MRI scan may be in response to an absence of electromagnetic fields and gaps for greater than a threshold period of time. In further examples, the completion of the MRI scan may be determined in response to a second sliding body motion while the patient is the generally horizontal body position (and which is in an opposite direction than the first sliding body motion), and which may be followed by further body motion while patient is in an upright or standing body position. Additionally, an electrical signal induced on the MRI-sensitive conductive element, Hall effect sensor, reed switch and/or magnetometer may decrease, indicating the patient is being removed from the bore of the MRI scanner.

As shown at 1290 of FIG. 35, the method may further comprise performing one or more of deactivating or activating a feature, switching a mode of operation, and performing a diagnostic test in response to the identified absence of the MRI system or completion of the MRI scan. For example, the IMD may switch from the MRI mode back to the normal-operation mode or may enable a therapy mode. In some examples, a diagnostics test is performed and the results may be communicated to external circuitry, such as the external device 26 illustrated by FIG. 2A. The diagnostics test may be used to verify the IMD is operating normally after the MRI scan and/or to identify and indicate a failure of the IMD.

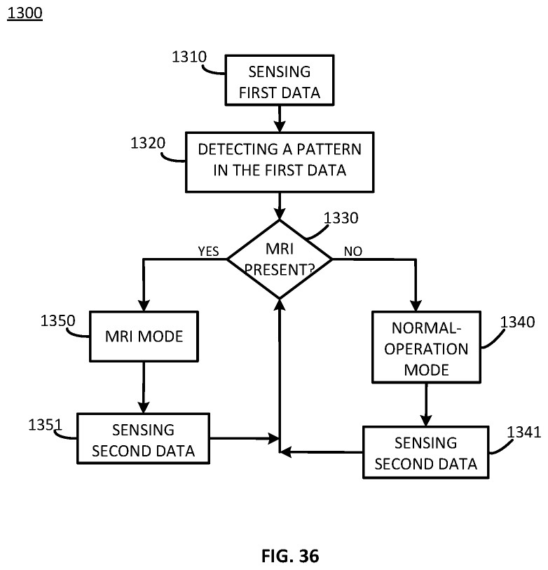

FIG. 36 is a diagram schematically representing an example method 1300, which may comprise part of a flow diagram in an example method (e.g., method 10). As shown at 1310, the method 1300 comprises sensing first data via at least one implantable sensor of an IMD system. The first data may comprise posture, motion, and vibrations sensed via an acceleration sensor. In some examples, the first data may further comprise static magnetic fields identified using a non-acceleration sensor. As shown at 1320, the method 1300 comprises determining a pattern in the first data that is indicative of a presence-absence state of the MRI system. In various examples, the identification of the presence-absence state may include identifying a probability of the presence of the MRI system, which is tracked over time. For example, as shown at 1330, the method 1300 includes identifying the presence of the MRI system based on the pattern, such as identifying the probability of the presence is greater than a threshold. If it is identified or determined that the MRI system is absent, as shown at 1340, the IMD may remain (or be placed) in a normal-operation mode. If it is identified or determined that the MRI system is present, as shown at 1350 the IMD is placed in an MRI mode (e.g., therapy is disabled or other care or device parameters are changed).

In either event, second data is sensed using the at least one implantable sensor, as shown at 1341 and/or at 1351. For example, the method 1300 may comprise monitoring for second data in response to the identified presence of the MRI system, as shown at 1351, and which may be used to verify the presence of the MRI system (e.g., increase the determined probability) and/or to identify the absence of the MRI system or completion of the MRI scan using the second data, and optionally, a data model. The second data may include externally induced vibration and/or electromagnetic fields indicative of a sequence of electromagnetic fields exerted by the MRI system, and identifying the absence of the MRI system includes identifying the vibrations and/or electromagnetic fields above one or more thresholds are absent for greater than a threshold period of time. The second data may additionally include at least one body motion and/or posture change, such as the second sliding body motion and movement to a standing position.

In such an example, as shown at 1330, an identification or determination is again made and the absence of the MRI system is identified and as shown at 1340, the normal-operation mode of the IMD is activated. In some examples, the method 1300 may comprise deactivating therapy of the IMD (e.g., preventing application of a stimulation signal) in response to the identified presence of the MRI system and activating therapy of the IMD in response to the absence or completion of the MRI scan. In some examples, activating the therapy may comprise delivering electrical simulation, via an implantable electrode of the IMD, to a nerve of the patient with the IMD implanted in response to detecting the patient is in a state of sleep based on third data sensed by the at least one implantable sensor. Although examples are not so limited, and the MRI mode may not include deactivation of therapy and/or the IMD may not provide therapy. For example, the MRI mode may include changing electronic configurations and/or other device settings.

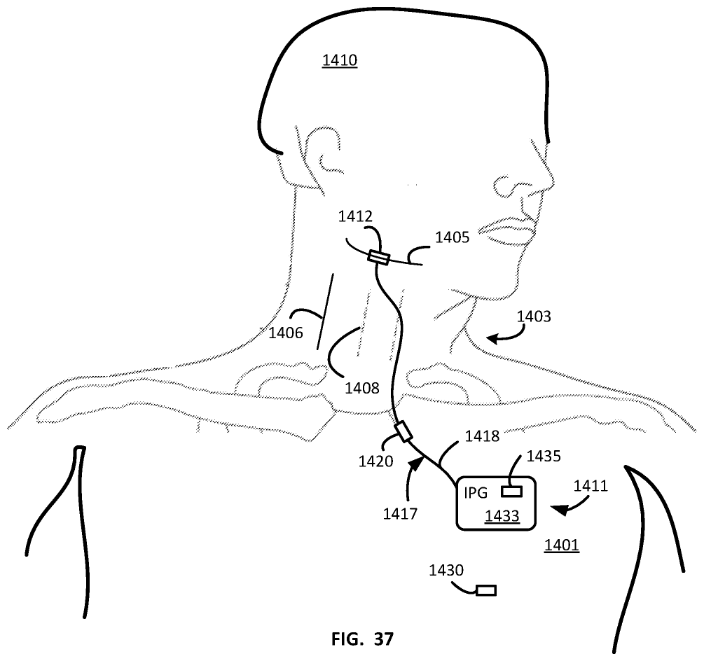

FIG. 37 is a diagram including a front view of an example device 1411 (and/or example method) implanted within a patient's body 1410. In some examples, the device 1411 may comprise an IMD such as (but not limited to) an implantable pulse generator (IPG) 1433 with IMD including a sensor 1435. In some examples, IMD 1411 comprises at least some of substantially the same features and attributes as IMD 22 (including the at least one implantable sensor 25), as previously described in association with at least FIG. 2A). Accordingly, in some examples, sensor 1435 may comprise at least an acceleration sensor (e.g., 25A in FIG. 3C, 110 in FIG. 5A, etc.) having at least some of substantially the same features and attributes as previously described in association with at least FIGS. 1-36. Via such example sensing arrangements, the IMD 1411 may identify the presence-absence state of an MRI system. For example, FIG. 37 illustrates an example IMD by which FIGS. 1-36 and/or FIGS. 39A-41 may be implemented.

As further shown in FIG. 37, device 1411 comprises a lead 1417 including a lead body 1418 for chronic implantation (e.g., subcutaneously via tunneling or other techniques) and to extend to a position adjacent a nerve (e.g., hypoglossal nerve 1405 and/or phrenic nerve 1406). The lead 1417 may comprise a stimulation electrode 1412 to engage the nerve (e.g., 1405, 1406) in a head-and-neck region 1403 for stimulating the nerve to treat a physiologic condition, such as sleep disordered breathing like obstructive sleep apnea, central sleep apnea, multiple-type sleep apneas, etc. The IMD 1411 may comprise circuitry, power element, etc. to support control and operation of both the sensor 1435 and the stimulation electrode 1412 (via lead 1417). In some examples, such control, operation, etc. may be implemented, at least in part, via a control portion (and related functions, portions, elements, engines, parameters, etc.) such as described later in association with at least FIGS. 39A-41.

With regard to the various examples of the present disclosure, in some examples, delivering stimulation to an upper airway patency nerve 1405 (e.g., a hypoglossal nerve) via the stimulation electrode 1412 is to cause contraction of upper airway patency-related muscles, which may cause or maintain opening of the upper airway (1408) to prevent and/or treat obstructive sleep apnea. Similarly, in some examples such electrical stimulation may be applied to a phrenic nerve 1406 via the stimulation electrode 1412 to cause contraction of the diaphragm as part of preventing or treating at least central sleep apnea. It will be further understood that some example methods may comprise treating both obstructive sleep apnea and central sleep apnea, such as but not limited to, instances of multiple-type sleep apnea in which both types of sleep apnea may be present at least some of the time. In some such instances, separate stimulation leads 1417 may be provided or a single stimulation lead 1417 may be provided but with a bifurcated distal portion with each separate distal portion extending to a respective one of the hypoglossal nerve 1405 and the phrenic nerve 1406.

In some such examples, the contraction of the hypoglossal nerve and/or contraction of the phrenic nerve caused by electrical stimulation comprises a suprathreshold stimulation, which is in contrast to a subthreshold stimulation (e.g., mere tone) of such muscles. In one aspect, a suprathreshold intensity level corresponds to a stimulation energy greater than the nerve excitation threshold, such that the suprathreshold stimulation may provide for higher degrees (e.g., maximum, other) of upper-airway clearance (i.e., patency) and sleep apnea therapy efficacy.

In some examples, a target intensity level of stimulation energy is selected, determined, implemented, etc. without regard to intentionally establishing a discomfort threshold of the patient (such as in response to such stimulation). Stated differently, in at least some examples, a target intensity level of stimulation may be implemented to provide the desired efficacious therapeutic effect in reducing SDB without attempting to adjust or increase the target intensity level according to (or relative to) a discomfort threshold.

In some examples, the treatment period (during which stimulation may be applied at least part of the time) may comprise a period of time beginning with the patient turning on the therapy device and ending with the patient turning off the device. In some examples, the treatment period may comprise a selectable, predetermined start time (e.g., 10 p.m.) and selectable, predetermined stop time (e.g., 6 a.m.). In some examples, the treatment period may comprise a period of time between an auto-detected initiation of sleep and auto-detected awake-from-sleep time. With this in mind, the treatment period corresponds to a period during which a patient is sleeping such that the stimulation of the upper airway patency-related nerve and/or central sleep apnea-related nerve is generally not perceived by the patient and so that the stimulation coincides with the patient behavior (e.g., sleeping) during which the sleep disordered breathing behavior (e.g., central or obstructive sleep apnea) would be expected to occur.

Information related to the treatment period, in various examples, may be input to the data model and/or otherwise used by the MRI engine to identify the presence-absence state of the MRI system. For example, if first data indicates a probability of the presence of an MRI system at night, the MRI engine may disregard and/or lower the probability as it is unlikely an MRI scan is occurring at night. Second data sensed, such as electromagnetic field patterns which are indicative of an MRI scan, may be used to further revise the probability.

Some non-limiting examples of such devices and methods to recognize and detect the various features and patterns associated with respiratory effort and flow limitations include, but are not limited to: U.S. Pat. No. 8,938,299, issued Jan. 20, 2015, and entitled “SYSTEM FOR TREATING SLEEP DISORDERED BREATHING”; U.S. Pat. No. 5,944,680, issued Aug. 31,1999, and entitled “RESPIRATORY EFFORT DETECTION METHOD AND APPARATUS”; and U.S. Pat. No. 5,522,862, issued Jun. 4, 1996, and entitled “METHOD AND APPARATUS FOR TREATING OBSTRUCTIVE SLEEP APNEA”, the entire teachings of each are hereby incorporated by reference herein in their entireties.

Moreover, in some examples various stimulation methods may be applied to treat obstructive sleep apnea, which include but are not limited to: U.S. Pat. No. 10,583,297, issued Mar. 10, 2020, and entitled “METHOD AND SYSTEM FOR APPLYING STIMULATION IN TREATING SLEEP DISORDERED BREATHING”; U.S. Pat. No. 8,938,299, issued Jan. 20, 2015, and entitled “SYSTEM FOR TREATING SLEEP DISORDERED BREATHING”; U.S. Pat. No. 5,944,680, issued Aug. 31, 1999, and entitled “RESPIRATORY EFFORT DETECTION METHOD AND APPARATUS”; and U.S. Patent Publication No. 2018/0117316, published May 3, 2018, and entitled “STIMULATION FOR TREATING SLEEP DISORDERED BREATHING”, the entire teachings of each are hereby incorporated by reference herein in their entireties.

In some examples, the example stimulation electrode(s) 1412 shown in FIG. 37 may comprise at least some of substantially the same features and attributes as described in: U.S. Pat. No. 8,340,785, issued on Dec. 25, 2012, and entitled “SELF EXPANDING ELECTRODE CUFF”; U.S. Pat. No. 9,227,053, issued on Jan. 5, 2016, and entitled “SELF EXPANDING ELECTRODE CUFF”; U.S. Pat. No. 8,934,992, issued on Jan. 13, 2015, and entitled “NERVE CUFF”; and U.S. Patent Publication No. 2020/0230412, published on Jul. 23, 2020, and entitled “CUFF ELECTRODE”, the entire teachings of which are each incorporated herein by reference in their entireties. Moreover, in some examples a stimulation lead 1417, which may comprise one example implementation of a stimulation element, may comprise at least some of substantially the same features and attributes as the stimulation lead described in U.S. Pat. No. 6,572,543, issued Jun. 3, 2003, and entitled “SENSOR, METHOD OF SENSOR IMPLANT AND SYSTEM FOR TREATMENT OF RESPIRATORY DISORDERS”, the entire teachings of which is incorporated herein by reference in its entirety. In other examples, stimulation elements include stimulation electrode(s) 1412 in different types of arrangements and/or for different targets, as previously described.

In some examples, the stimulation electrode 1412 may be delivered transvenously, percutaneously, etc. In some such examples, a transvenous approach may comprise at least some of substantially the same features and attributes as described in U.S. Pat. No. 9,889,299, issued Feb. 13, 2018, entitled “TRANSVENOUS METHOD OF TREATING SLEEP APNEA”, and which is hereby incorporated by reference in its entirety. In some such examples, a percutaneous approach may comprise at least some of substantially the same features and attributes as described in U.S. Pat. No. 9,486,628, issued Nov. 8, 2016, and entitled “PERCUTANEOUS ACCESS FOR SYSTEMS AND METHODS OF TREATING SLEEP APNEA”, the entire teachings of which is incorporated herein by reference in its entirety.

As further shown in the diagram of FIG. 37, in some examples device 1411 may be implemented with additional sensors 1420, 1430 to sense additional physiologic data, such as but not limited to, further respiratory information via sensing transthoracic bio-impedance, pressure sensing, etc. in order to complement the respiration information sensed via an acceleration sensor. In some examples, one or both of the sensors 1420, 1430 may comprise sensor electrodes. In some examples, stimulation electrode 1412 also may act, in some examples, as a sensing electrode. In some examples, at least a portion of housing of the IPG 1433 also may comprise a sensor or at least an electrically conductive portion (e.g., electrode) to work in cooperation with sensing electrodes to implement at least some sensing arrangements to sense bioimpedance, ECG, etc.

However, examples are not so limited and may be directed to other neurostimulation devices and cardiac care devices which may detect cardiac signals and provide atrial chamber stimulation therapy. For example, the IMD may include or be coupled to an implantable leads using to sense left and right atrial and ventricular cardiac signals. The electronics assembly of the IMD processes or monitors the cardiac signals and provides stimulation signals using a pulse generator and the implantable leads.

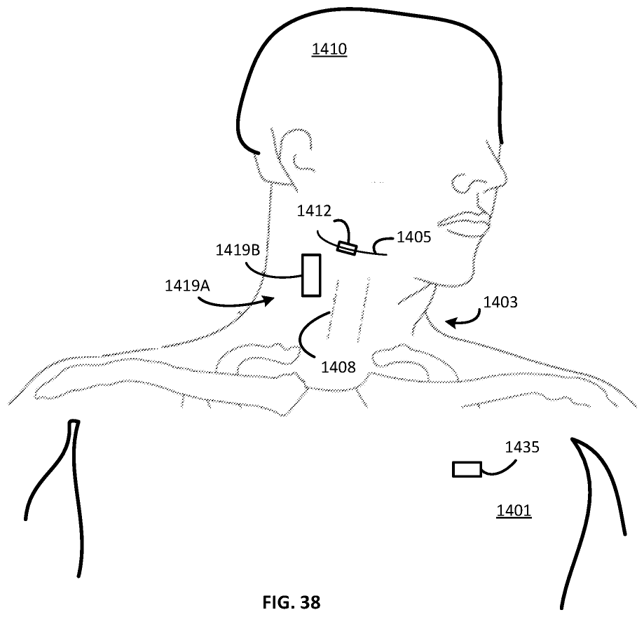

FIG. 38 is a diagram schematically representing an example IMD 1419A comprising at least some of substantially the same features and attributes as the IMD 1411 in FIG. 37, except with the IPG 1433 implemented as a microstimulator 1419B. In some examples, the microstimulator 1419B may be chronically implanted (e.g., percutaneously, subcutaneously, transvenously, etc.) in a head-and-neck region 1403 as shown in FIG. 38, or in a pectoral region 1401. In some examples, as part of the IMD 1419A, the microstimulator 1419B may be in wired or wireless communication with stimulation electrode 1412. In some examples, as part of the IMD 1419A, the microstimulator 1419B also may incorporate sensor 1435 or be in wireless or wired communication with a sensor 1435 located separately from a body of the microstimulator 1419B. When wireless communication is employed for sensing and/or stimulation, the microstimulator 1419B may be referred to as leadless implantable medical device for purposes of sensing and/or stimulation. In some examples, the microstimulator 1419B may be in close proximity to a target nerve 1405.

In some examples, the microstimulator 1419B (and associated elements) and/or IMD 1419A may comprise at least some of substantially the same features and attributes as described and illustrated in U.S. Patent Publication No. 2020/0254249, filed on Aug. 8, 2020, and entitled “MICROSTIMULATION SLEEP DISORDERED BREATHING (SDB) THERAPY DEVICE”, the entire teachings of which is incorporated herein by reference in its entirety.

As implicated by the above description, one or both of the IMD and the external device includes a controller, control unit, or control portion that prompts performance of designated actions. FIG. 39A is a block diagram schematically representing an example control portion 1600. In some examples, the control portion 1600 includes a controller 1602 and a memory 1604. In some examples, the control portion 1600 provides one example implementation of a control portion forming a part of, implementing, and/or managing any one of devices, systems, assemblies, circuitry, managers, engines, functions, parameters, sensors, electrodes, modules, and/or methods, as represented throughout the present disclosure in association with FIGS. 1-38.

In general terms, the controller 1602 of the control portion 1600 comprises an electronics assembly 1606 (e.g., at least one processor, microprocessor, integrated circuits and logic, etc.) and associated memories or storage devices. The controller 1602 is electrically couplable to, and in communication with, the memory 1604 to generate control signals to direct operation of at least some the devices, systems, assemblies, circuitry, managers, modules, engines, functions, parameters, sensors, electrodes, and/or methods, as represented throughout the present disclosure. In some examples, these generated control signals include, but are not limited to, employing the MRI engine 27 of an IMD which may be a software program stored on the memory 1604 (which may be stored on another storage device and loaded onto the memory 1604), and executed by the electronics assembly 1606 to at least identify the presence-absence state of an MRI system. In addition, and in some examples, these generated control signals include, but are not limited to, employing the care engine 1610 stored in the memory 1604 to at least manage care provided to the patient, for example cardiac therapy or therapy for sleep disordered breathing, in at least some examples of the present disclosure. It will be further understood that the control portion 1600 (or another control portion) may also be employed to operate general functions of the various care devices/systems described throughout the present disclosure.

In response to or based upon commands received via a user interface (e.g., user interface 1640 in FIG. 40) and/or via machine readable instructions, controller 1602 generates control signals as described above in accordance with at least some of the examples of the present disclosure. In some examples, controller 1602 is embodied in a general purpose computing device while in some examples, controller 1602 is incorporated into or associated with at least some of the sensors, sensing element, MRI identification elements, respiration determination elements, stimulation elements, power/control elements (e.g., pulse generators), devices, user interfaces, instructions, information, engines, functions, actions, and/or method, etc. as described throughout examples of the present disclosure.

For purposes of this application, in reference to the controller 1602, the term “processor” shall mean a presently developed or future developed processor (or processing resources) that executes machine readable instructions contained in a memory. In some examples, execution of the machine readable instructions, such as those provided via memory 1604 of control portion 1600 cause the processor to perform the above-identified actions, such as operating controller 1602 to implement the sensing, monitoring, identifying the presence-absence state of an MRI system, stimulation, treatment, etc. as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by memory 1604. In some examples, the machine readable instructions may comprise a sequence of instructions, a processor-executable machine learning model, or the like. In some examples, memory 1604 comprises a computer readable tangible medium providing non-volatile storage of the machine readable instructions executable by a process of controller 1602. In some examples, the computer readable tangible medium may sometimes be referred to as, and/or comprise at least a portion of, a computer program product. In some examples, hard wired circuitry may be used in place of or in combination with machine readable instructions to implement the functions described. For example, controller 1602 may be embodied as part of at least one application-specific integrated circuit (ASIC), at least one field-programmable gate array (FPGA), and/or the like. In at least some examples, the controller 1602 is not limited to any specific combination of hardware circuitry and machine readable instructions, nor limited to any particular source for the machine readable instructions executed by the controller 1602.

In some examples, control portion 1600 may be entirely implemented within or by a stand-alone device.

In some examples, the control portion 1600 may be partially implemented in one of the sensors, sensing element, MRI identification elements, respiration determination elements, monitoring devices, stimulation devices, IMDs (or portions thereof), etc. and partially implemented in a computing resource (e.g., at least one external resource) separate from, and independent of, the IMDs (or portions thereof) but in communication with the IMDs (or portions thereof). For instance, in some examples control portion 1600 may be implemented via a server accessible via the cloud and/or other network pathways. In some examples, the control portion 1600 may be distributed or apportioned among multiple devices or resources such as among a server, an apnea treatment device (or portion thereof), and/or a user interface.

In some examples, control portion 1600 includes, and/or is in communication with, a user interface 1640 as shown in FIG. 40.

FIG. 39B is a diagram schematically illustrating at least some example arrangements of a control portion 1620 by which the control portion 1600 (FIG. 39A) may be implemented. In some examples, control portion 1620 is entirely implemented within or by an IPG assembly 1625, which has at least some of substantially the same features and attributes as a pulse generator (e.g., power/control element) as previously described throughout the present disclosure. In some examples, control portion 1620 is entirely implemented within or by a remote control 1630 (e.g., a programmer) external to the patient's body, such as a patient control 1632 and/or a physician control 1634. In some examples, the control portion 1600 is partially implemented in the IPG assembly 1625 and partially implemented in the remote control 1630 (at least one of patient control 1632 and physician control 1634).

FIG. 40 is a block diagram schematically representing a user interface 1640. In some examples, user interface 1640 forms part of and/or is accessible via a device external to the patient and by which the IMD system may be at least partially controlled and/or monitored. The external device which hosts user interface 1640 may be a patient remote (e.g., 1632 in FIG. 39B), a physician remote (e.g., 1634 in FIG. 39B) and/or a clinician portal. In some examples, user interface 1640 comprises a user interface or other display that provides for the simultaneous display, activation, and/or operation of at least some of the sensors, sensing element, respiration determination elements, stimulation elements, power/control elements (e.g., pulse generators), devices, user interfaces, instructions, information, modules, engines, functions, actions, and/or method, etc., as described in association with FIGS. 1-40B. In some examples, at least some portions or aspects of the user interface 1640 are provided via a graphical user interface (GUI), and may comprise a display 1644 and input 1642.

FIG. 41 is a block diagram 1650 which schematically represents some example implementations by which an IMD 1660 (e.g., IMD 22 (e.g., an IPG), implantable sensing monitor, and the like) may communicate wirelessly with external devices outside the patient. As described above, the controller and/or control portion of the IMD 1660 illustrated in FIG. 41 may be implemented by components of the IMD 1660, components of external devices (e.g., mobile device 1670, patient remote control 1674, a clinician programmer 1676, and a patient management tool 1680), and various combinations thereof. As shown in FIG. 41, in some examples, the IMD 1660 may communicate with at least one of patient application 1672 on a mobile device 1670, a patient remote control 1674, a clinician programmer 1676, and a patient management tool 1680. The patient management tool 1680 may be implemented via a cloud-based portal 1683, the patient application 1672, and/or the patient remote control 1674. Among other types of data, these communication arrangements enable the IMD 1660 to communicate, display, manage, etc. the identified presence-absence state of the MRI system, data collected during and after the MRI system (e.g., logged events, data patterns, and diagnostic results), as well as to allow for adjustment to the various elements, portions, etc. of the example devices and methods if and where desired. In some examples, the various forms of identified presence-absence state of the MRI system be displayed to a patient and/or clinician via one of the above-described external devices. The displayed information may comprise each of the identified presence-absence state of the MRI system, data sensed to identify the presence and to identify the absence, patterns identified and associated probabilities, logged events during the presence of the MRI system, and IMD device diagnostic results. Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

Various examples are implemented in accordance with the underlying Provisional Application Ser. No. 63/089,118, entitled “IDENTIFYING A PRESENCE-ABSENCE STATE OF A MAGNETIC RESONANCE IMAGING SYSTEM,” filed Oct. 8, 2020, to which benefit is claimed and which is fully incorporated herein by reference for its general and specific teachings. For instance, examples herein and/or in the Provisional Application can be combined in varying degrees (including wholly). Reference can also be made to the experimental teachings and underlying references provided in the underlying Provisional Application. Examples discussed in the Provisional Application are not intended, in any way, to be limiting to the overall technical disclosure, or to any part of the claimed disclosure unless specifically noted.

1-156. (canceled)

157. A method comprising:
sensing first data via at least one implantable sensor of an implantable medical device (IMD) system; and
identifying a presence-absence state of a magnetic resonance imaging (MM) system using the first data.

158. The method of claim 157, wherein identifying the presence-absence state of the MRI system comprises applying a data model to the first data to identify at least one pattern within the first data indicative of the presence-absence state of the MRI system.

159. The method of claim 157, wherein identifying the presence-absence state of the MRI system comprises assessing at least one of a probability of a presence of the MRI system and a probability of the absence of the MRI system.

160. The method of claim 157, wherein identifying the presence-absence state of the MRI system comprises applying a data model to the first data to identify at least one pattern comprising a sequence of motion, a sequence of vibrations, a sequence of electrical signals induced on internal electronics, and a sequence of electromagnetic fields the IMD system is exposed to.

161. The method of claim 160, wherein the pattern comprises a sequence of electromagnetic fields comprising at least one of:
time-varying gradient magnetic fields;
static magnetic fields;
radio frequency (RF) fields; and
gaps between the electromagnetic fields, the gaps comprising periods of time without the time-varying gradient magnetic fields and the RF fields.

162. The method of claim 157, wherein identifying the presence-absence state comprises applying a data model to the first data and second data to identify at least one pattern indicative of a presence of the MM system within the first data and the second data.

163. The method of claim 157, wherein the first data comprises vibration data and identifying the presence-absence state of the MRI system comprises identifying a pattern of vibration using the vibration data.

164. The method of claim 157, wherein the at least one implantable sensor comprises an acceleration sensor and the first data comprises motion data, and identifying the presence-absence state of the MM system comprises identifying a pattern of body motion indicative of initiation of an MM scan by the MM system.

165. The method of claim 157, wherein the at least one implantable sensor comprises an acceleration sensor.

166. The method of claim 165, further comprising detecting a sliding body motion while a patient with the IMD implanted is in a supine position using the first data sensed by the acceleration sensor and in response, identifying the presence-absence state of the MM system.

167. The method of claim 157, wherein the at least one implantable sensor comprises an acceleration sensor and an MRI-sensitive conductive element, and sensing the first data comprises sensing at least one of motion and electromagnetic fields via the acceleration sensor and the MM-sensitive conductive element.

168. The method of claim 157, further comprising constructing a data model to identify the presence-absence state of the MM system via known inputs corresponding to at least one of a motion pattern and an electromagnetic field pattern relative to known outputs corresponding to the presence-absence state of the MRI system.

169. The method of claim 157, wherein identifying the presence-absence state of the MRI system comprises tracking a probability of a presence of the MRI system.

170. The method of claim 157, further comprising disabling a feature of the IMD in response to the identified presence-absence state of the MM system.

171. The method of claim 157, further comprising disabling a sleep detection feature of the IMD in response to the identified presence-absence state of the MRI system comprising a presence of the MM system.

172. The method of claim 157, further comprising transitioning the IMD to an MM mode in response to the identified presence-absence state of the MM system comprising a presence of the MM system.

173. The method of claim 157, further comprising in response to the identified presence-absence state of the MM system, performing at least one of:
logging the presence-absence state of the MRI system;
logging events of the IMD; and
communicating logged events to external circuitry.

174. The method of claim 157, further comprising performing a diagnostics test on the IMD in response the identified presence-absence state of the MM system or completion of an MRI scan, and communicating results of the diagnostics test to external circuitry.

175. The method of claim 157, wherein the presence-absence state comprises a presence of the MM system, the method further comprising obtaining second data and identifying at least one of an absence of the MM system and completion of an MRI scan using the second data and a data model.

176. The method of claim 157, further comprising sensing, via the at least one implantable sensor, at least one of:
time-varying gradient magnetic fields sensed during the MM scan; and
static magnetic fields sensed responsive to a patient with the IMD implanted moving into the MRI system.
